(19) United States
(12) Patent Application Publication
(10) Pub. No.: US 2024/0390474 A1
(43) Pub. Date: Nov. 28, 2024

(54) COMPOSITIONS AND METHODS FOR THERAPEUTIC OR VACCINE DELIVERY

(71) Applicant: GenVivo, Inc., San Marino, CA (US)

(72) Inventors: Jacqueline FISCHER-LOUGHEED, Duarte, CA (US); Bradford H. STEELE, Arcadia, CA (US); Cecilia ROH, South Pasadena, CA (US); Robert G. JOHNSON, JR., Lafayette, CA (US)

(21) Appl. No.: 18/646,575

(22) Filed: Apr. 25, 2024

Related U.S. Application Data
(60) Provisional application No. 63/498,772, filed on Apr. 27, 2023.

(51) Int. Cl. A61K 39/12; A61K 39/00; C12N 7/00; C12N 15/86

(52) U.S. Cl. CPC A61K 39/12; C12N 7/00; C12N 15/86; A61K 2039/5254; A61K 2039/575; C12N 2740/10022; C12N 2740/10043; C12N 2770/36134

(57) ABSTRACT

Described herein are compositions comprising recombinant viral vectors, e.g., recombinant retroviral vectors, for delivering a therapeutic or a vaccine. The recombinant retroviral vectors described herein are modified for safer application of therapeutic or vaccine delivery. Also described herein are methods for using the compositions comprising recombinant viral vectors for delivering a therapeutic or a vaccine.

### CROSS-REFERENCE

This application claims the benefit of U.S. Provisional Application No. 63/498,772 filed on Apr. 27, 2023, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

The use of genetic material such as viral vectors for delivering therapeutics has emerged as one of the foundations of modern medicine. However, such genetic material can lead to undesired mitogenic effects. For example, retroviral vectors may integrate into genomes of cells, with a risk of transforming such cells into cancer cells. One remedy for such problem is to use RNA-based vehicle for delivering genetic material.

### SUMMARY

The use of RNA-based vehicles can be complex and laborious. Additionally, RNA-based vehicles are prone to degradation and may exhibit a short half-life. Other DNA-based vehicles (e.g., non-viral DNA) for delivering therapeutics face challenges including efficiency of transporting and targeting the vehicle to target cells and induction of immune response or toxicity in the subject. Accordingly, there remains a need for a vehicle for delivering therapeutics such as viral vectors, e.g., retroviral vectors, for delivering therapeutics without integrating genetic materials into the genome of host cells.

Described herein, in some aspects, is a recombinant retroviral vector comprising a first nucleic acid sequence having a deleted integrase and a second nucleic acid sequence encoding at least one payload, said deleted integrase, when compared to a wild-type integrase, is a deletion of about 12%, about 15%, about 20%, about 30%, about 40%, about 50%, about 60%, about 70%, about 80%, about 90%, about 95%, about 99% or about 100% of the integrase coding sequence (SEQ ID NO: 6); and said at least one payload comprises an antigen. In some embodiments, the deleted integrase is a deletion of at least 80, 160, 240, 320, or 408 residues of amino acids 1331-1738 of a murine leukemia virus Gag-Pol polyprotein (SEQ ID NO: 1). In some embodiments, the deleted integrase does not have retroviral integration activity. In some embodiments, the antigen comprises a pathogen polypeptide or fragment thereof or a cancer polypeptide or fragment thereof. In some embodiments, the cancer polypeptide or fragment thereof is associated with a cancer cell or a tumor microenvironment. In some embodiments, the pathogen polypeptide or fragment thereof comprises a viral polypeptide or fragment thereof. In some embodiments, the pathogen polypeptide or fragment thereof comprises a bacterium polypeptide or fragment thereof. In some embodiments, the pathogen polypeptide or fragment thereof comprises a fungus polypeptide or fragment thereof. In some embodiments, the pathogen polypeptide or fragment thereof comprises a protist polypeptide or fragment thereof. In some embodiments, the pathogen polypeptide or fragment thereof comprises a protozoa polypeptide or fragment thereof. In some embodiments, the viral polypeptide or fragment thereof comprises a coronavirus polypeptide or fragment thereof. In some embodiments, the coronavirus polypeptide or fragment thereof comprises a Severe Acute Respiratory Syndrome (SARS-CoV) polypeptide or fragment thereof, a SARS-CoV-2 polypeptide or fragment thereof, or a Middle East Respiratory Syndrome (MERS-CoV) polypeptide or fragment thereof. In some embodiments, the coronavirus polypeptide or fragment thereof comprises the SARS-CoV-2 polypeptide or fragment thereof. In some embodiments, the SARS-CoV-2 polypeptide or fragment thereof comprises a Spike protein or fragment thereof. In some embodiments, the Spike protein or fragment thereof is a full-length Spike protein. In some embodiments, the Spike protein or fragment thereof is a truncated Spike protein. In some embodiments, the truncated Spike protein comprises N-terminal domain of the Spike protein or S2 domain of the Spike protein. In some embodiments, the truncated Spike protein comprises N-terminal domain of the Spike protein and S2 domain of the Spike protein. In some embodiments, the Spike protein or fragment thereof is a recombinant Spike protein. In some embodiments, the Spike protein or fragment thereof comprises at least one modification. In some embodiments, the at least one modification comprises codon optimization. In some embodiments, the codon optimization optimizes or increases expression of the at least one payload in a human cell. In some embodiments, the at least one modification comprises at least one amino acid mutation. In some embodiments, the at least one amino acid mutation eliminates a cleavage site in the Spike protein. In some embodiments, the cleavage site is a furin cleavage site. In some embodiments, the furin cleavage site comprises amino acid residues 682-685, amino acid residues 679-682, or amino acid residue 811 in SEQ ID NO: 21 or SEQ ID NO: 22. In some embodiments, the cleavage site is a serine protease cleavage site. In some embodiments, the serine protease cleavage site comprises amino acid residues 986 and 987; or 983 and 984 in SEQ ID NO: 21 or SEQ ID NO: 22. In some embodiments, the at least one amino acid mutation comprises amino acid substitution at amino acid residue 814, 889, 896, 939, 682-685, 679-682, 811, 986, 987, 983, 984, or a combination thereof in SEQ ID NO: 21 or SEQ ID NO: 22. In some embodiments, the at least one amino acid mutation comprises amino acid substitution at amino acid residue 814, 889, 896, and 939 in SEQ ID NO: 21 or SEQ ID NO: 22. In some embodiments, the Spike protein or fragment thereof comprises a signal peptide. In some embodiments, the signal peptide is a secretory peptide. In some embodiments, the signal peptide is a membrane targeting peptide. In some embodiments, the signal peptide comprises an amino acid sequence of SEQ ID NO: 13: MDAMKRGLCCVLLLCGAVFVSASQEIHARFRR. In some embodiments, the secretory peptide comprises an amino acid sequence that is at least 70% identical to IgE Fc receptor alpha. In some embodiments, the viral polypeptide or fragment thereof comprises an influenza polypeptide or fragment thereof. In some embodiments, the influenza polypeptide or fragment thereof comprises an influenza A polypeptide or fragment thereof, an influenza B polypeptide or fragment thereof, an influenza C polypeptide or fragment thereof, or an influenza D polypeptide or fragment thereof. In some embodiments, the influenza polypeptide or fragment thereof is the influenza A polypeptide or fragment thereof. In some embodiments, the influenza A polypeptide or fragment thereof comprises neuraminidase (NA) or fragment thereof or hemagglutinin (HA) or fragment thereof. In some embodiments, the influenza A polypeptide or fragment thereof comprises the hemagglutinin (HA) or fragment thereof. In some embodiments, the hemagglutinin (HA) or fragment thereof is a full-length hemagglutinin (HA). In some embodiments, the hemagglutinin (HA) or fragment thereof is a truncated hemagglutinin (HA). In some embodiments, the truncated hemagglutinin (HA) comprises Stalk domain. In some embodiments, the hemagglutinin (HA) or fragment thereof is a recombinant hemagglutinin (HA). In some embodiments, the hemagglutinin (HA) or fragment thereof comprises at least one modification. In some embodiments, the at least one modification comprises codon optimization. In some embodiments, the codon optimization optimizes or increases expression of the at least one payload in a human cell. In some embodiments, the at least one modification comprises at least one amino acid mutation. In some embodiments, the at least one modification comprises the hemagglutinin (HA) or fragment thereof comprising an amino acid sequence of extracellular domain of M2 protein (M2e) of influenza A: SEQ ID NO: 14: MSLLTEVETPIRNEWGCRCNDSSD. In some embodiments, the recombinant retroviral vector encodes at least one envelope protein. In some embodiments, the at least one envelope protein comprises at least one alphavirus envelope protein. In some embodiments, the at least one alphavirus envelope protein comprises at least one Sindbis virus envelope protein. In some embodiments, the at least one Sindbis virus envelope protein comprises E3 protein, E2 protein, 6K protein, E1 protein, or a combination thereof. In some embodiments, the at least one Sindbis virus envelope protein comprises at least one mutation. In some embodiments, the at least one mutation increases binding affinity between the at least one Sindbis virus envelope protein and a human cell. In some embodiments, the human cell is a dendritic cell. In some embodiments, the at least one mutation is E160G of the E2 protein. In some embodiments the wildtype E2 protein has a sequence at least 90%, 95%, 99%, or 100% identical to SEQ ID NO: 26. In some embodiments, the at least one envelope protein comprises at least one rhabdovirus envelope protein. In some embodiments, the at least one rhabdovirus envelope protein comprises a vesicular stomatitis virus (VSV) envelope protein. In some embodiments, the at least one envelope protein comprises a retrovirus envelope protein. In some embodiments, the at least one retrovirus envelope protein comprise a murine leukemia virus envelope protein or a Moloney murine leukemia virus envelope protein. In some embodiments, the recombinant retroviral vector comprises at least one modified untranslated region (UTR). In some embodiments, the at least one modified UTR comprises a 5′-UTR. In some embodiments, the at least one modified UTR comprises a 3′-UTR. In some embodiments, the at least one modified UTR comprises a 5′-UTR and a 3′-UTR.

Described herein, in some aspects, is a recombinant virus comprising a recombinant retroviral vector described herein. In some embodiments, the recombinant virus is a recombinant Sindbis virus. In some embodiments, the recombinant Sindbis virus comprises E160G mutation of the E2 protein. In some embodiments the wildtype E2 protein has a sequence at least 90%, 95%, 99%, or 100% identical to SEQ ID NO: 26.

Described herein, in some aspects, is a cell comprising a recombinant retroviral vector described herein or a recombinant virus of described herein. In some embodiments, the cell expresses the at least one payload. In some embodiments, the cell secretes the at least one payload. In some embodiments, the cell expresses and secretes the at least one payload. In some embodiments, the cell expresses the at least one payload for at least one day, at least three days, at least five days, or at least nine days. In some embodiments, the cell secretes the at least one payload for at least one day, at least three days, at least five days, or at least nine days. In some embodiments, the cell expresses and secretes the at least one payload for at least one day, at least three days, at least five days, or at least nine days.

Described herein, in some aspects, is a pharmaceutical composition comprising a recombinant retroviral vector described herein, a recombinant virus described herein, or a cell comprising a recombinant retroviral vector described herein or a recombinant virus described herein. In some embodiments, the pharmaceutical composition comprises at least one additional active ingredient. In some embodiments, the at least one additional active ingredient comprises an adjuvant. In some embodiments, the pharmaceutical composition comprises at least one pharmaceutically acceptable excipient.

Described herein, in some aspects, is a method of treating or preventing a disease or condition in a subject, comprising administering to the subject a recombinant retroviral vector described herein, a recombinant virus described herein, a cell comprising a recombinant retroviral vector described herein or a recombinant virus described herein, or a pharmaceutical composition described herein, wherein the at least one payload comprising the antigen induces immune response in the subject, thereby treating or preventing the disease or condition in the subject. In some embodiments, the immune response comprises induction of neutralizing antibody targeting the antigen, thereby generating immunity against the antigen in the subject. In some embodiments, the immune response comprises induction of immunoglobulin antibody targeting the antigen, thereby generating immunity against the antigen in the subject. In some embodiments, the immunoglobulin antibody comprises IgG antibody, IgM antibody, IgA antibody, IgD antibody, IgE antibody, or a combination thereof. In some embodiments, the immunoglobulin antibody comprises IgG antibody. In some embodiments, the at least one payload is expressed in the subject for at least one day, at least three days, at least five days, or at least nine days. In some embodiments, the at least one payload is secreted in the subject for at least one day, at least three days, at least five days, or at least nine days. In some embodiments, a duration of the immune response induced by the at least one payload is expressed for at least one day, at least three days, at least five days, or at least nine days is increased by at least 10%, at least 20%, at least 50%, at least 100%, at least 5-fold, at least 10-fold, or more compared to a duration of an immune response induced by a comparable payload that is expressed for fewer than one day, three days, five days, or nine days. In some embodiments, a duration of the immune response induced by the at least one payload is secreted for at least one day, at least three days, at least five days, or at least nine days is increased by at least 10%, at least 20%, at least 50%, at least 100%, at least 5-fold, at least 10-fold, or more compared to a duration of an immune response induced by a comparable payload that is expressed for fewer than one day, three days, five days, or nine days. In some embodiments, the immune response persists in the subject for at least three months, at least four months, at least five months, at least six months, at least 12 months, or longer.

Described herein, in some aspects, is a method of treating a disease or condition in a subject, comprising administering a therapeutically effective amount of a pharmaceutical composition comprising the recombinant retroviral vector described herein to the subject.

Described herein, in some aspects, is a method of vaccinating a subject, comprising administering a pharmaceutical composition comprising the recombinant retroviral vector described herein to the subject.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows the integrase is part of the gagpol gene which is translated into a polyprotein sequence that gets processed by the viral protease to yield 7 proteins. The integrase (INT) is a 408 aa protein at the C-Terminus of Gag-Pol.

FIG. 2A shows a plasmid map of WT-GP.

FIG. 2B shows a plasmid map of GP-IntDel after the integrase deletion.

FIG. 3A shows HSV1-TK (vTK) expression detected by FACS (Fluorescence-Activated Cell Sorting) on Day 2, Day 3, Day 7, and Day 14 post-transduction with vectors with WT-GP, GP-IntDel, or GP-Integrase Defective IDRV2-2. IntDel behaves similarly to GP-Integrase-defective IDRV2-2 although expression is reduced compared to WT-GP. NTDs are control non-transduced cells.

FIG. 3B shows vTK/Ganciclovir (GCV) cell kill activity on Day 3, Day 7, and Day 14 post-transduction with vectors with WT-GP, GP-IntDel, or GP-Integrase Defective IDRV2-2. Earlier days of cell kill activities are comparable among different GP vectors.

FIG. 4 shows copGFP expression detected in A375 cells by FACS on Day 2, Day 3, Day 7, Day 10, and Day 14 post-transduction with vectors with WT-GP, GP-IntDel, or IDRV mutants. Particularly, IDRV1 is D184A, IDRV2 is D125AD184A, and IDRV2-2 is D184AE220A. NTDs are control non-transduced cells.

FIGS. 5A-5C show payload HSV1-TK protein expression detected by FACS over time post-transduction in A375 melanoma cells (FIG. 5A), A431 epidermoid carcinoma cells (FIG. 5B) and A459 lung carcinoma cells (FIG. 5C). These cells were transduced with vectors generated with GPwt, GP-Integrase-defective IDRV2, and IntDel. IntDel behaves similarly to GP-Integrase-defective IDRV2 although expression is reduced compared to GP-Integrase-defective IDRV2. NTDs are control non-transduced cells.

FIGS. 6A-6D show HSV1-TK/GCV cell kill activity over time post-transduction of A375 cells (FIG. 6A), A431 cells (FIG. 6B), A549 cells (FIG. 6C), and U87MG malignant brain glioma cells (FIG. 6D) with vectors generated with GPwt, or IntDel, or GP-Integrase-defective IDRV2.

FIG. 7 shows Western blot detection of expression of SARS-CoV2 Omicron Spike protein secreted over time by A375 cells transduced with vectors made with GPwt, GP-Integrase-defective IDRV2, and IntDel, respectively.

FIGS. 8A-8C show lack of payload gene integration into host genome by ddPCR (FIG. 8A) and disappearance of payload RNA over time by PCR (FIGS. 8B-8C) in A375 transduced with vectors made with GPwt, IntDel, and GP-Integrase-defective IDRV2. FIG. 8C is an expansion of FIG. 8B without the GPwt to show that there are similar levels of Psi copies/μg RNA in IDRV2 and IntDel and that they both disappear over time, as expected.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Therapeutic delivery using vectors, e.g., recombinant viral vectors, is one of the fastest-growing areas in medicine. A wide range of strategies are being investigated to improve targeting, efficiency, and safety of this approach. Recombinant viral vectors, e.g., recombinant retroviral vectors, can be used as a delivery vehicle to a transfer payload, e.g., a therapeutic, into a target cell. Wild-type retroviral gagpol plasmids (e.g., SEQ ID NO: 4) comprise a nucleic acid sequence encoding several proteins, such as integrase, which is an enzyme that integrates viral genetic information into the genome of a host cell. This can, in some instances, affect normal function of the host cell or increase a risk of transforming the host cell into cancer cells.

Described herein, in some aspects, is a vector comprising at least one amino acid mutation. In some aspects, the vector is a recombinant retroviral vector (or a retrovector) comprising the at least one amino acid mutation, where the at least one amino acid mutation is a deletion of the integrase encoded by the recombinant retroviral vector, thus creating a deleted integrase. In one aspect, the present disclosure provides a recombinant retroviral vector comprising a first nucleic acid sequence having a deleted integrase and a second nucleic acid sequence encoding at least one payload. In some aspects, the deleted integrase does not have retroviral integration activity and can no longer integrate the recombinant retroviral vector genome into genome of the host cell. In some embodiments, the vector, e.g., recombinant retroviral vector, comprises a nucleic acid construct comprising at least one nucleic acid sequence encoding a mutant reverse transcriptase compared to a wild-type reverse transcriptase (SEQ ID NO: 2). In some aspects, the mutant reverse transcriptase comprises at least one amino acid mutation, where the mutant reverse transcriptase can no longer convert the vector into DNA for inserting into the genome of the host cell. In some embodiments, the vector, e.g., recombinant retroviral vector, comprises a nucleic acid construct comprising at least one nucleic acid sequence encoding both the deleted integrase and the mutant reverse transcriptase.

In some aspects, the recombinant retroviral vector comprising a nucleic acid construct comprising a nucleic acid sequence having a deleted integrase, wherein the deleted integrase, compared with a wild-type integrase (SEQ ID NO: 1), comprises a deletion of about 12%, about 15%, about 20%, about 30%, about 40%, about 50%, about 60%, about 70%, about 80%, about 90%, about 95%, about 99%, or about 100% of the integrase coding sequence. In embodiments, the mutant integrase is a deletion of at least about 80, about 160, about 240, about 320, or about 408 residues of amino acids 1331-1738 of a murine leukemia virus Gag-Pol polyprotein.

In another aspect, the present disclosure provides recombinant viruses comprising a recombinant retroviral vectors as described herein. In some aspects, the present disclosure provides a cell comprising a recombinant retroviral vector or a recombinant virus as described herein. In one aspect, the present disclosure provides a pharmaceutical composition comprising a recombinant retroviral vector, a recombinant virus, or a cell as described herein. In another aspects, the present disclosure provides a method of treating or preventing a disease or condition in a subject, wherein the method comprising administering to the subject a recombinant retroviral vector, a recombinant virus, a cell as described herein, or a pharmaceutical composition comprising a recombinant retroviral vector, a recombinant virus, or a cell as described herein.

In some aspects, the vector comprising the deleted integrase can also encode a payload. In some embodiments, the payload comprises a therapeutic. In some embodiments, the vector described herein comprises a recombinant retroviral vector comprising the payload encoding an antigen, where the antigen stimulates innate immunity (e.g., as a vaccine). In some embodiments, the antigen comprises a pathogen polypeptide or fragment thereof, or a cancer polypeptide or fragment thereof. In some embodiments, the cancer polypeptide or fragment thereof is associated with a cancer cell or a tumor microenvironment. In some embodiments, the pathogen polypeptide or fragment thereof comprises a virus polypeptide or fragment thereof. In some embodiments, the virus polypeptide or fragment thereof comprises a coronavirus polypeptide or fragment thereof. In some embodiments, the virus polypeptide or fragment thereof comprises an influenza polypeptide or fragment thereof. In some embodiments, the pathogen polypeptide or fragment thereof comprises a bacterium polypeptide or fragment thereof. In some embodiments, the pathogen polypeptide or fragment thereof comprises a fungus polypeptide or fragment thereof. In some embodiments, the pathogen polypeptide or fragment thereof comprises a protist polypeptide or fragment thereof. In some embodiments, the pathogen polypeptide or fragment thereof comprises a protozoa polypeptide or fragment thereof.

In some embodiments, the payload comprises an antigen, where the antigen can elicit immune response, thus vaccinating a subject administered with the vector. In some aspects, described herein is a method of treating a disease or condition in a subject by administering the vector (e.g., the recombinant retroviral vector described herein) to the subject, where the vector delivers a therapeutic as payload of the vector. In some aspects, described herein is a method of vaccinating a subject by administering the vector (e.g., the recombinant retroviral vector described herein) to the subject, where the vector delivers an antigen as payload. The antigen can then trigger innate immune response against the antigen, thus vaccinating the subject.

### Vector

Described herein, in some aspects, is a vector such as a recombinant retroviral vector comprising a nucleic acid construct comprising at least one nucleic acid sequence having a deleted integrase such that the vector does not have retroviral integration activity.

In some embodiments, the vector, e.g., recombinant retroviral vector, comprises a nucleic acid construct comprising at least one nucleic acid sequence encoding a mutant reverse transcriptase. In some embodiments, the vector, e.g., recombinant retroviral vector, comprises a nucleic acid construct comprising at least one nucleic acid sequence encoding both the deleted integrase and the mutant reverse transcriptase.

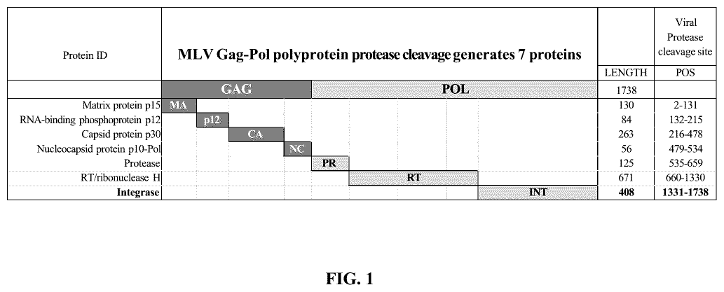

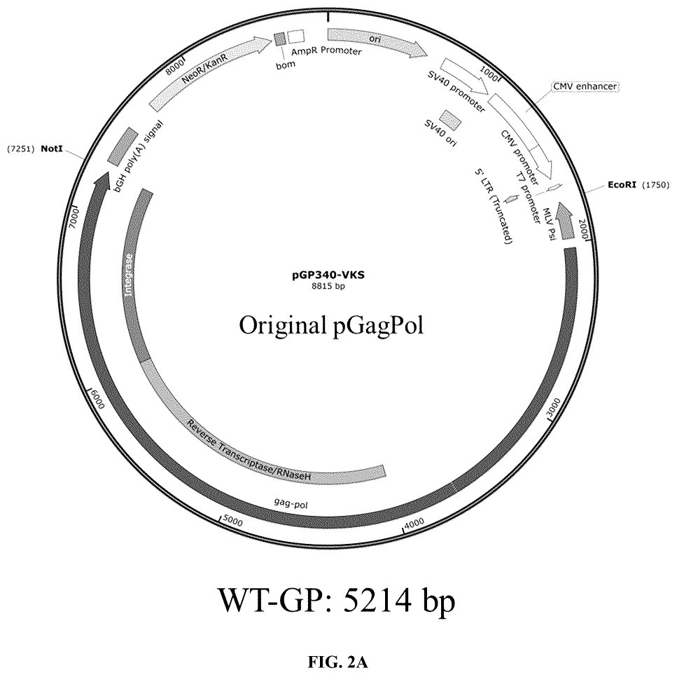

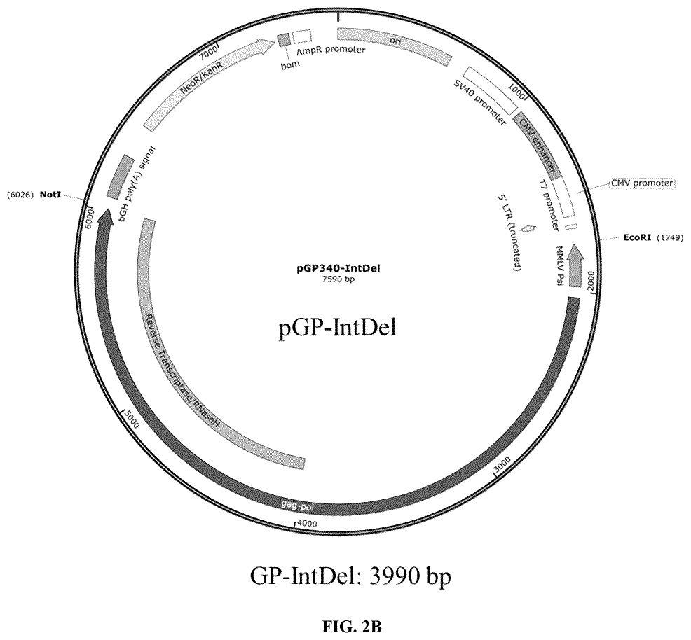

In some embodiments, the deleted integrase, compared to a wild-type integrase (SEQ ID NO: 5), is a deletion of about 12%, about 15%, about 20%, about 30%, about 40%, about 50%, about 60%, about 70%, about 80%, about 90%, about 95%, about 99%, or about 100% of the integrase coding sequence (SEQ ID NO: 6). FIG. 2A shows a plasmid diagram of a wild-type murine leukemia virus (MLV) Gag-Pol polyprotein, while FIG. 2B illustrates an exemplary plasmid diagram showing the deleted integrase. As shown in FIG. 1, MLV Gag-Pol polyprotein comprises 7 proteins, which are matrix protein p15, RNA-binding phosphoprotein p12, capsid protein p30, nucleocapsid protein p10-Pol, protease, RT/ribonuclease H, and integrase. Length and protease cleavage site of each protein are indicated accordingly. Also shown in FIG. 1, integrase is a 408 amino acid protein at the C-Terminus of the Gag-Pol, from position 1331-1738 of the Gag-Pol. In some embodiments, the deleted integrase is a deletion of at least about 80, about 160, about 240, about 320, or about 408 residues of amino acids 1331-1738 of a murine leukemia virus Gag-Pol polyprotein.

In some embodiments, the vector, e.g., recombinant retroviral vector comprising the deleted integrase comprises a deletion of about 12%, about 15%, about 20%, about 30%, about 40%, about 50%, about 60%, about 70%, about 80%, about 90%, about 95%, about 99%, or about 100% of an amino acid sequence of wild type MLV Gag-Pol Polyprotein (UniProt Access number: P03355POL_MLVMS) (SEQ ID NO: 1). In some embodiments, the vector, e.g., recombinant retroviral vector comprising the deleted integrase comprises a deletion of about 12%, about 15%, about 20%, about 30%, about 40%, about 50%, about 60%, about 70%, about 80%, about 90%, about 95%, about 99%, or about 100% of a nucleic acid sequence of wild type MLV Gag-Pol (SEQ ID NO: 2). In some embodiments, a nucleic acid sequence of wild type Moloney murine leukemia virus comprises a nucleic acid sequence with NCBI Reference Sequence: NC_001501.

In some embodiments, the vector, e.g., recombinant retroviral vector comprising the deleted integrase comprises a deletion of about 12%, about 15%, about 20%, about 30%, about 40%, about 50%, about 60%, about 70%, about 80%, about 90%, about 95%, about 99%, or about 100% of an amino acid sequence of wild type integrase (SEQ ID NO: 5). In some embodiments, the vector, e.g., recombinant retroviral vector comprising the deleted integrase comprises a deletion of about 12%, about 15%, about 20%, about 30%, about 40%, about 50%, about 60%, about 70%, about 80%, about 90%, about 95%, about 99%, or about 100% of a nucleic acid sequence of wild type integrase (SEQ ID NO: 6).

In some embodiments, the vector, e.g., recombinant retroviral vector, comprising the deleted integrase comprises a nucleic acid sequence of SEQ ID NO: 4. In some embodiments, the vector, e.g., recombinant retroviral vector, comprising the deleted integrase comprises an amino acid sequence of SEQ ID NO: 3. In SEQ ID NO: 1 and SEQ ID NO: 3, the asterisk (*) represents a stop codon, which is located between the end of the p10 nucleocapsid and the start of the protease, and is counted as one of the amino acid residues.

In some embodiments, the vector, e.g., recombinant retroviral vector, comprising the deleted integrase renders the integrase dysfunctional such that the vector does not have retroviral integration activity. In some embodiments, the vector comprising the deleted integrase can no longer introduce the genome or genetic materials of the vector, e.g., a recombinant retroviral vector, into genome of a host cell.

In some embodiments, the vector, e.g., the recombinant retroviral vector, comprises at least one promoter for expressing the at least one nucleic acid sequence. For example, the vector comprises a CMV promoter for expressing the at least one nucleic acid sequence encoding a payload, such as an interleukin (e.g., the P40 subunit and the P35 subunit of interleukin-12 (IL-12). In some aspects, the promoter comprises a muscle specific promoter such as HSA (human skeletal a-actin promoter), a muscle creatine kinase (MCK)-gene based promoter such as CK6 or MHCK7 promoter; a Desmin gene promoter (DES); a constitutive human promoter EF-1α (Elongation Factor 1α). Non-limiting examples of promoters are the retroviral LTR; the SV40 promoter; the Rous Sarcoma Virus (RSV) promoter; the histone promoter; the polIII promoter, the β-actin promoter; inducible promoters, such as the MMTV promoter, the metallothionein promoter, and heat shock promoters; adenovirus promoters; the albumin promoter; the ApoAI promoter; B19 parvovirus promoters; human globin promoters; viral thymidine kinase promoters, such as the Herpes Simplex Virus thymidine kinase promoter; retroviral LTRs; human growth hormone promoters; and the MxIFN inducible promoter. In some embodiments, the promoter is a tissue-specific promoter. In some embodiments, a tissue specific promoter is chosen from the group including, but not limited to, the tyrosinase related promoters (TRP-1 and TRP-2), DF3 enhancer (for breast cells), SLPI promoter (secretory leucoprotease inhibitor-expressed in many types of carcinomas), TRS (tissue specific regulatory sequences), α-fetoprotein promoters (specific for normal hepatocytes and transformed hepatocytes, respectively), the carcino-embryonic antigen promoter (for use in transformed cells of the gastrointestinal tract, lung, breast and other tissues), the tyrosine hydroxylase promoter (for melanocytes), choline acetyl transferase or neuron specific enolase promoters for use in neuroblastomas, the regulatory sequence for glial fibroblastomas, the tyrosine hydroxylase promoter, c-erb B-2 promoter, PGK promoter, PEPCK promoter, whey acidic promoter (breast tissue), and casein promoter (breast tissue), and the adipocyte P2 promoter. In some embodiments, the promoter is a viral promoter (e.g., retroviral promoters, as well as others such as HIV promoters), hepatitis promoters, herpes promoters (e.g., EBV). In some embodiments, the promoter is the native HSV-TK promoter. In some embodiments, the promoter is a bacterial, fungal or parasitic (e.g., malarial)-specific promoter utilized in order to target a specific cell or tissue which is infected with a virus, bacteria, fungus or parasite. In some aspects, the vector comprises a nucleic acid sequence for encoding a tag such as His tag or a Flag tag for purification, imaging, or expression control purposes.

In some aspects, the vector is a viral vector such as a retroviral vector. Viral vectors, and especially retroviral vectors, e.g., recombinant retroviral vectors, have become the most widely used method for inserting genes into mammalian, e.g., human, cells. Other viral vectors, in some embodiments, are derived from lentivirus, poxviruses, herpes simplex virus I, adenoviruses, adeno-associated viruses, or Sindbis viruses. Non-limiting examples of viral vectors are retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the recombinant retroviral vectors include, but are not limited to, gamma-retroviral vectors such as vectors derived from the Moloney Murine Leukemia Virus (MoMLV, MMLV, MuLV, or MLV) or the Murine Stem cell Virus (MSCV) genome. In some instances, the recombinant retroviral vectors also include, but are not limited to, lentiviral vectors such as those derived from the human immunodeficiency virus (HIV) genome. In some instances, AAV vectors are selected from AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

In some embodiments, the recombinant retroviral vector comprises at least one envelope protein. In some embodiments, the at least one envelope protein is an amphotropic envelope. In some embodiments, the recombinant retroviral vector comprises the at least one envelope protein comprising the amphotropic envelope. In some embodiments, the at least one envelope protein is a modified envelope protein, e.g., mutation of the wild-type envelope protein or conjugation with targeting moiety, to increase targeting specificity or transduction efficiency.

In some embodiments, the recombinant retroviral vector is a pseudotyped viral vector. In viral pseudotyping, viral particles or viral vectors can be generated using viral envelope proteins from another virus to either restrict or broaden the host cell range. In some embodiments, the at least one envelope protein comprises at least one alphavirus envelope protein. In some embodiments, at least one alphavirus envelope protein comprises at least one Sindbis virus envelope protein. In some embodiments, the at least one Sindbis virus envelope protein comprises E3 protein, E2 protein, 6K protein, E1 protein, or a combination thereof. In some embodiments, the at least one Sindbis virus envelope protein comprises at least one mutation. In some embodiments, the at least one mutation increases binding affinity between the at least one Sindbis virus envelope protein and a target cell or a host cell such as a human cell, e.g., a dendritic cell. In some embodiments, the at least one mutation of the at least one Sindbis virus envelope protein is E160G of the E2 protein. In some embodiments the wildtype E2 protein has a sequence at least 90%, 95%, 99%, or 100% identical to SEQ ID NO: 26. In some embodiments, the at least one envelope protein is a vesicular stomatitis virus envelope protein.

In some embodiments, the recombinant retroviral vector comprises at least one modification, wherein the recombinant retroviral vector comprises at least one amino acid mutation for increasing targeting efficiency of the recombinant retroviral vector to a cell type. For example, a recombinant retroviral vector pseudotyped with Sindbis virus envelope protein can comprise an E160G mutation in E2 protein of the Sindbis virus envelope protein. In some embodiments the wildtype E2 protein has a sequence at least 90%, 95%, 99%, or 100% identical to SEQ ID NO: 26. In some instances, the E160G mutation increases targeting of the Sindbis virus envelope protein to a dendritic cell. In such, the targeting of the dendritic cell can increase immune response and the efficacy of vaccination by contacting the dendritic cell with an antigen encoded by the recombinant retroviral vector.

In some embodiments, the vector, e.g., recombinant retroviral vector, encodes a targeting moiety such as an antibody for targeting a cell surface marker (e.g., an antigen expressed on the cell surface of a cell associated with the disease or condition). In some embodiments, the cell surface marker is a tumor-associated antigen such as Her2. In some embodiments, the targeting moiety comprises an antibody, a nanobody (e.g., a single-chain variable fragment or scFv), or a combination thereof. In some embodiments, the targeting moiety is expressed on the surface of the viral envelope, where the vector described herein is encapsulated in the viral envelope. In some embodiments, the targeting moiety increases the targeting or delivering of the vector to the cell or microenvironment associated with the disease or condition such as cancer or lesion.

In some embodiments, the vector, e.g., a recombinant retroviral vector described herein, comprises at least one modified untranslated region (UTR). In some embodiments, the at least one UTR is a 5′-UTR located at 5′ end of the nucleic acid sequence of the at least one payload. In some embodiments, the at least one UTR is a 3′-UTR located at 3′ end of the nucleic acid sequence of the at least one payload. In some embodiments, the at least one UTR comprises both a 5′-UTR and a 3′-UTR located at both 5′ end and 3′ end of the nucleic acid sequence of the at least one payload. In some embodiments, the at least one modified UTR increases expression of the at least one payload compared to where the at least one payload is flanked by a wild-type UTR.

In some aspects, the vector, e.g., recombinant retroviral vector, comprising the deleted integrase further comprises at least one payload. Described herein, in some aspects, is a recombinant retroviral vector comprising a first nucleic acid sequence having a deleted integrase described herein and a second nucleic acid sequence encoding at least one payload. In some embodiments, the payload comprises a therapeutic or an antigen. As described herein, a therapeutic refers to an agent that can be utilized to treat, modulate, or improve a disease or condition. Examples of therapeutic include, but are not limited to, a nucleic acid sequence, a nucleic acid molecule, a polypeptide or a fragment thereof, a cytokine, an enzyme, or a combination thereof.

In some embodiments, the at least one payload comprises a nucleic acid sequence, a nucleic acid molecule, a polypeptide or a fragment thereof, a cytokine, an interferon, an enzyme, or a combination thereof. In some embodiments, the at least one payload comprises a nucleic acid sequence encoding a transgene, a protein, an antigen, a polypeptide or a fragment thereof, a cytokine, or an enzyme.

In some embodiments, the at least one payload comprises a nucleic acid molecule. In some instances, the nucleic acid molecule can be used to modulate a gene expression of a target cell. In some instances, the nucleic acid molecule can alter a gene expression of a target cell. In some instances, the nucleic acid molecule can increase a gene expression of a target cell. In some instances, the nucleic acid molecule can decrease a gene expression of a target cell. Nucleic acid molecules that can be delivered to an individual using vectors described herein include, but are not limited to, DNA, RNA, or combination thereof. In some instances, the nucleic acid molecule comprises a non-translated RNA, an oligonucleotide, an antisense RNA, a ribozyme, an RNAi, an miRNA, an shRNA, or an siRNA. In some embodiments, the nucleic acid molecule comprises heterogenous nucleic acids.

In some embodiments, the vector, e.g., recombinant retroviral vector, comprising the deleted integrase comprises at least one therapeutic or at least one antigen. In some embodiments, the at least one therapeutic comprises a cytokine. In some embodiments, cytokine comprises an interleukin or an interferon. In some embodiments, the vector comprises at least one nucleic acid sequence encoding at least one interleukin subunit. In some embodiments, the vector comprises at least one nucleic acid sequence encoding at least two interleukin subunits, wherein the at least two interleukin subunits are the same. In some embodiments, the vector comprises at least one nucleic acid sequence encoding at least two interleukin subunits, wherein the at least two interleukin subunits are different. In some embodiments, the vector comprises at least one nucleic acid sequence encoding one interleukin subunit. In some embodiments, the vector comprises at least one nucleic acid sequence encoding two interleukin subunits. In some embodiments, the vector comprises at least one nucleic acid sequence encoding two different interleukin subunits. In some embodiments, the vector comprises at least one nucleic acid sequence encoding two or more different interleukin subunits. In some embodiments, the vector, e.g., recombinant retroviral vector, comprises at least one start codon for expressing the interleukin, the subunit of the interleukin, or a combination thereof. In some embodiments, the vector, e.g., recombinant retroviral vector, comprises at least two start codons for expressing two interleukins, two subunits of the interleukin, or a combination thereof. In some embodiments, the vector, e.g., recombinant retroviral vector, comprises two codons each for expressing an interleukin subunit. Non-limiting examples of the interleukin can include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, IL-40, or IL-41. In some embodiments, the interleukin comprises IL-7. In some embodiments, the interleukin comprises IL-12.

In some embodiments, the vector, e.g., recombinant retroviral vector, comprising the deleted integrase comprises a therapeutic comprising an interferon. In some embodiments, the vector, e.g., recombinant retroviral vector, comprises at least one nucleic acid sequence encoding an interferon. In some embodiments, the interferon comprises IFNα, IFNβ, IFNγ, or a combination thereof.

In some embodiments, the vector, e.g., recombinant retroviral vector, described herein comprises at least one payload. In some embodiments, the payload comprises a nucleic acid sequence encoding an antigen. In some embodiments, the antigen can stimulate immunity (e.g., as a vaccine). In some embodiments, the antigen comprises a pathogen polypeptide or fragment thereof or a cancer polypeptide or fragment thereof. In some embodiments, the cancer polypeptide or fragment thereof is associated with a cancer cell or a tumor microenvironment. In some embodiments, the cancer polypeptide or fragment thereof is associated with cancer. In some embodiments, the cancer comprises leukemia, myeloblastic, promyelocytic, myelomonocytic, monocytic, erythroleukemia, chronic myelocytic (granulocytic) leukemia, and chronic lymphocytic leukemia, lymphoma, e.g. Hodgkin's disease and non-Hodgkin's disease, fibrosarcoma, myosarcoma, liposarcoma, chondrosarcoma, osteogenic sarcoma, angio-sarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuro-blastoma, retinoblastoma, dysplasia and hyperplasia, prostate cancer, prostatitis, benign prostatic hypertrophy, benign prostatic hyperplasia (BPH), prostatic paraganglioma, prostate adenocarcinoma, prostatic intraepithelial neoplasia, rectal-prostate fistulas, and atypical prostatic stromal lesions. In some instances, the cancer comprises melanoma, epidermoid carcinoma, lung cancer, or glioblastoma.

In some embodiments, the pathogen polypeptide or fragment thereof comprises a viral polypeptide or fragment thereof. In some embodiments, the viral polypeptide or fragment thereof comprises a coronavirus polypeptide or fragment thereof. In some embodiments, the viral polypeptide or fragment thereof comprises an influenza polypeptide or fragment thereof.

In some embodiments, the pathogen polypeptide or fragment thereof comprises a bacterium polypeptide or fragment thereof. In some embodiments, the pathogen polypeptide or fragment thereof comprises a fungus polypeptide or fragment thereof. In some embodiments, the pathogen polypeptide or fragment thereof comprises a protist polypeptide or fragment thereof. In some embodiments, the pathogen polypeptide or fragment thereof comprises a protozoa polypeptide or fragment thereof.

In some embodiments, the at least one payload comprises an antigen. In some embodiments, the antigen induces immune response in a cell. In some embodiments, the antigen comprises a pathogen polypeptide. In some embodiments, the pathogen polypeptide includes, but is not limited to, a polypeptide derived from a bacterial pathogen, an alveolate pathogen, an amoebal pathogen, a fungal pathogen, a protozoan pathogen, a nematodal pathogen, a platyhelminthes pathogen, a viral pathogen, or a combination thereof. Table 1 provides a non-limiting example list for such pathogen(s) as described above, which may serve as a basis for the design of nucleotide sequences encoding pathogen-derived polypeptides for incorporation into retroviral vector(s) for transduction and eventual expression as antigenic payloads by transduced cells.

TABLE 1
List of pathogens
Pathogen

| Class | Genus | Member |
|---|---|---|
| Bacterial | Listeria | Listeria monocytogenes |
| | Bordetella | Bordetella brionchiseptica |
| | | Bordatella pertussis |
| | | Bordatella parapertussis 1 |
| | | Bordatella parapertussis 2 |
| | Rickettsia | Rickettsia rickettsii |
| | | Rickettsia akari |
| | | Rickettsia conorii |
| | | Rickettsia sibirica |
| | | Rickettsia australis |
| | | Rickettsia felis |
| | | Rickettsia japonica |
| | | Rickettsia africae |
| | | Rickettsia hoogstraalii |
| | | Rickettsia prowazekii |
| | | Rickettsia typhi |
| | Orientia | Orientia tsutsugamushi |
| | | Orientia chuto |
| | Piscireckettsia | Piscirickettsia salmonis |
| | Corynebacterium | Corynebacterium diptheria |
| | | Corynebacterium ulcerans |
| | Streptococcus | Streptococcus aureus |
| | | Streptococcus pyrogenes |
| | | Streptococcus pneumoniae |
| | | Streptococcus agalactiae (Group B Strep) |
| | Clostridium | Clostridium botulinum |
| | | Clostridium perfringens |
| | | Clostridium difficle |
| | | Clostridium tetani |
| | Borrelia | Borrelia turcica |
| | | Borrelia coriaceae |
| | | Borrelia miyamotoi |
| | | Borrelia mayonii |
| | | Borrelia americana |
| | | Borrelia lanei |
| | | Borrelia turdi |
| | | Borrelia tanukii |
| | | Borrelia californiensis |
| | | Borrelia kurtenbachii |
| | | Borrelia yangtzensis |
| | | Borrelia valaisiana |
| | | Borrelia afzelii |
| | | Borrelia spielmanii |
| | | Borrelia maritima |
| | | Borrelia lusitaniae |
| | | Borrelia garinii garinii |
| | | Borrelia garinii bavariensis |
| | | Borrelia japonica |
| | | Borrelia sinica |
| | | Borrelia burgdorferi |
| | | Borrelia bissettii |
| | | Borrelia carolinensis |
| | Leptospira | Leptospira alexanderi |
| | | Leptospira borgpetersenii |
| | | Leptospira interrogans |
| | | Leptospira mayottensis |
| | | Leptospira kirchneri |
| | | Leptospira noguchii |
| | | Leptospira santorosai |
| | | Leptospira weilii |
| | Mycobacterium | Mycobacterium tuberculosis |
| | | Mycobacterium africanum |
| | | Mycobacterium orygis |
| | | Mycobacterium bovis |
| | | Mycobacterium microti |
| | | Mycobacterium canetti |
| | | Mycobacterium caprae |
| | | Mycobacterium pinnipedii |
| | | Mycobacterium suricattae |
| | | Mycobacterium mungi |
| | Vibrio | Vibrio vulnificus |
| | | Vibrio cholerae |
| | Bacillus | Bacillus anthracis |
| | Neorickettsia | Neorickettsia risticii |
| | Proteus | Proteus hauseri |
| | | Proteus mirabilis |
| | | Proteus myxofaciens |
| | | Proteus penneri |
| | | Proteus vulgaris |
| | Serratia | Serratia marcescens |
| | | Serratia plymuthica |
| | | Serratia liquefaciens |
| | | Serratia rubidaea |
| | | Serratia odorifera |
| | | Serratia fonticola |
| | Yersinia | Yersinia pestis |
| | | Yersina enterocolitica |
| | | Yersinia pseudotuberculosis |
| | | Yersenia Ruckerii |
| | Klebsiella | Klebsiella aerogenes |
| | | Klebsiella granulomatis |
| | | Klebsiella grimontii |
| | | Klebsiella huaxiensis |
| | | Klebsiella kielensis |
| | | Klebsiella michiganensis |
| | | Klebsiella milletis |
| | | Klebsiella oxytoca |
| | | Klebsiella pneumoniae |
| | | Klebsiella quasipneumoniae |
| | | Klebsiella similipneumoniae |
| | | Klebsiella quasivariicola |
| | | Klebsiella senegalensis |
| | | Klebsiella steroids |
| | | Klebsiella variicola |
| | Enterobacter | Enterobacter cloacae |
| | | Enterobacter cancerogenous |
| | | Enterobacter aerogenes |
| | | Enterobacter hormaechei |
| | Pluralibacter | Pluralibacter gergoviae |
| | Citrobacter | Citrobacter diversus |
| | | Citrobacter freundii |
| | | Citrobacter amalonaticus |
| | | Citrobacter braakii |
| | | Citrobacter farmeri |
| | | Citrobacter koseri |
| | | Citrobacter sedlakii |
| | | Citrobacter werkmanii |
| | | Citrobacter Youngae |
| | Salmonella | Salmonella bongori |
| | | Salmonella enterica subsp. Enterica |
| | | Salmonella choleraesuis |
| | | Salmonella dublin |
| | | Salmonella enteritidis |
| | | Salmonella gallinarum |
| | | Salmonella hadar |
| | | Salmonella heidelberg |
| | | Salmonella infantis |
| | | Salmonella paratyphi |
| | | Salmonella typhi |
| | | Salmonella typhimurium |
| | Shigella | Shigella boydii |
| | | Shigella dysenteriae |
| | | Shigella flexneri |
| | | Shigella sonnei |
| | Escherichia | Escherichia albertii |
| | | Escherichia coli |
| | | Escherichia fergusonii |
| | | Escherichia hermannii |
| | | Escherichia marmotae |
| | | Escherichia vulneris |
| Alveolate | Plasmodium | Plasmodium falciparum |
| | | Plasmodium knowlesi |
| | | Plasmodium vivax |
| | | Plasmodium ovale wallikeri |
| | | Plasmodium ovale curtisi |
| | | Plasmodium malariae |
| | Toxoplasma | Toxoplasma gondii |
| Amoebal | Naegleria | Naegleria fowleri |
| | Entabloeba | Entamoeba hystolytica |
| Fungal | Blastomyces | Blastomyces dermatitidis |
| | | Blasomyces gilchristi |
| | | Blastomyces helicus |
| | | Blastomyces percursus |
| | | Blastomyces emzantsi |
| | Coccidioides | Coccidioides immitis |
| | | Coccidioides posadasii |
| | Histoplasma | Histoplasma capsulatum |
| | Pneumocystis | Pneumocystis jirovecii |
| | Talaromyces | Talaromyces marneffei |
| | Sporothrix | Sporothrix schenkii |
| Protozoan | Leishmania | Leishmania aethiopica |
| | | Leishmania amazonensis |
| | | Leishmania arabica |
| | | Leishmania archibaldi |
| | | Leishmania aristedesi |
| | | Leishmania (Viannia) braziliensis |
| | | Leishmania chagasi |
| | | Leishmania donovani |
| | | Leishmania (Mundinia) enriettii |
| | | Leishmania forattinii |
| | | Leishmania garnhami |
| | | Leishmania gerbili |
| | | Leishmania (Viannia) guyanensis |
| | | Leishmania infantum |
| | | Leishmania killicki |
| | | Leishmania (Viannia) lainsoni |
| | | Leishmania major |
| | | Leishmania (Mundinia) macropodum |
| | | Leishmania (Mundinia) martiniquensis |
| | | Leishmania mexicana |
| | | Leishmania (Viannia) naiffi |
| | | Leishmania (Viannia) panamensis |
| | | Leishmania (Viannia) peruviana |
| | | Leishmania pifanoi |
| | | Leishmania (Viannia) shawi |
| | | Leishmania tarentolae |
| | | Leishmania tropica |
| | | Leishmania turanica |
| | | Leishmania waltoni |
| | | Leishmania venezuelensis |
| | Trypanosoma | Trypanosoma brucei |
| | | Trypanosoma rhodesiense |
| | | Trypanosoma equiperdum |
| | | Trypanosoma vivax |
| | | Trypanosoma congolense |
| | | Trypanosoma cruzi |
| | | Trypanosoma lewisi |
| | | Trypanosoma melophagium |
| | | Trypanosoma nabiasi |
| | | Trypanosoma rangeli |
| | | Trypanosoma theileri |
| | | Trypanosoma theodori |
| | | Trypanosoma cruzi |
| | | Trypanosoma cruzi marinkellei |
| | | Trypanosoma dionisii |
| | | Trypanosoma erneyi |
| | | Trypanosoma livingstonei |
| | | Trypanosoma wauwau |
| | | Trypanosoma conorhini |
| | | Trypanosoma rangeli |
| | Giardia | Giardia duodenalis |
| Nematodal | Onchocerca | Onchocerca volvulus |
| Platyhelminthes | Taenia | Taenia solium (pork) |
| | | Taenia saginata (beef) |
| | Diphyllobothrium | Diphyllobothrium latum |
| | | Diphyllobothrium dendriticum |
| | | Diphyllobothrium nihonkainense/klebanovskii |
| | Schistoma | Schistosoma bomfordi |
| | | Schistosoma bovis |
| | | Schistosoma curassoni |
| | | Schistosoma datta |
| | | Schistosoma edwardiense |
| | | Schistosoma guineensis |
| | | Schistosoma haematobium |
| | | Schistosoma harinasutai |
| | | Schistosoma hippopotami |
| | | Schistosoma incognitum |
| | | Schistosoma indicum |
| | | Schistosoma intercalatum |
| | | Schistosoma japonicum |
| | | Schistosoma kisumuensis |
| | | Schistosoma leiperi |
| | | Schistosoma malayensis |
| | | Schistosoma mansoni |
| | | Schistosoma margrebowiei |
| | | Schistosoma mattheei |
| | | Schistosoma mekongi |
| | | Schistosoma ovuncatum |
| | | Schistosoma nasale |
| | | Schistosoma rodhaini |
| | | Schistosoma sinensium |
| | | Schistosoma spindale |
| | | Schistosoma turkestanicum |
| Arthropod | Sarcoptes | Sarcoptes scabiei |
| Viral | Hepatovirus | Hepatitis A |
| | Orthohepadnavirus | Hepatitis B |
| | Deltavirus | Deltavirus italiense [HDV-1] |
| | | Deltavirus japanense [HDV-2] |
| | | Deltavirus peruense [HDV-3] |
| | | Deltavirus taiwanense [HDV-4] |
| | | Deltavirus togense [HDV-5] |
| | | Deltavirus carense [HDV-6] |
| | | Deltavirus cameroonense [HDV-7] |
| | | Deltavirus senegalense [HDV-8] |
| | Orthoherpesvirus | Orthoherpesvirus A (Hepatitis E) |
| | Alphavirus | Aura virus |
| | | Barmah Forest virus |
| | | Bebaru virus |
| | | Caaingua virus |
| | | Cabassou virus |
| | | Chikungunya virus |
| | | Eastern equine encephalitis virus |
| | | Eilat virus |
| | | Everglades virus |
| | | Fort Morgan virus |
| | | Getah virus |
| | | Highlands J virus |
| | | Madariaga virus |
| | | Mayaro virus |
| | | Middelburg virus |
| | | Mosso das Pedras virus |
| | | Mucambo virus |
| | | Ndumu virus |
| | | O'nyong'nyong virus |
| | | Pixuna virus |
| | | Rio Negro virus |
| | | Ross River virus |
| | | Salmon pancreas disease virus |
| | | Semliki Forest virus |
| | | Sindbis virus |
| | | Southern elephant seal virus |
| | | Tonate virus |
| | | Trocara virus |
| | | Una virus |
| | | Venezuelan equine encephalitis virus |
| | | Western equine encephalitis virus |
| | | Whataroa virus |
| | Novirhabdovirus | infectious hematopoetic necrosis virus (salmonid norvirhabdovirus) |
| | Gammaretrovirus | Feline leukemia virus |
| | Protoparvovirus | Feline panleukopenia virus |
| | | Canine parvovirus |
| | | Mink enteritis virus |
| | Deltaretrovirus | Bovine leukemia virus |
| | | Human T-cell lymphotropic virus |
| | Betacoronovirus/Embecovirus | Bovine coronavirus |
| | | Human coronavirus OC43 |
| | | China Rattus coronavirus HKU24 |
| | | Human coronavirus HKU1 |
| | | Murine coronavirus |
| | | Myodes coronavirus 2JL14 |
| | Betacoronovirus/Sarbecovirus | Severe Acute Respiratory Syndrome Coronavirus 1 |
| | | Severe Acute Respiratory Syndrome Coronavirus 2 |
| | | Bat SARS-like coronavirus WIV1 |
| | | Bat coronavirus RaTG13 |
| | Betacoronovirus/Merbecovirus | Middle East Respiratory Syndrome-Related Coronavirus |
| | | Pipistrellus bat coronavirus HKU5 |
| | | Tylonycteris bat coronavirus HKU4 |
| | Betacoronovirus/Nobecovirus | Eidolon bat coronavirus C704 |
| | | Rousettus bat coronavirus GCCDC1 |
| | | Rousettus bat coronavirus HKU9 |
| | Betacoronovirus/Hibbecovirus | Bat Hp-betacoronavirus Zhejiang2013 |
| | Paramyxovirus | Sosuga pararubulavirus |
| | Henipavirus | Hendra hinipivirus |
| | | Nipah virus |
| | Arenavirus | Lassa virus |
| | Ebolavirus | Ebola virus |
| | | Bundibugyo virus |
| | | Sudan virus |
| | | Taï Forest virus |
| | | Reston virus |
| | | Bombali virus |
| | Marburgvirus | Marburgh virus |
| | | Ravn virus |
| | Flavivirus | West Nile virus |
| | | Zika virus |
| | | Dengue virus |
| | | Yellow fever virus |
| | | Japanese encephalitis virus |
| | | Hepatitis C |
| | | Tick-borne encephalitis virus (and subtypes) |
| | Rotavirus | Rotavirus A |
| | | Rotavirus B |
| | | Rotavirus C |
| | | Rotavirus D |
| | | Rotavirus F |
| | | Rotavirus G |
| | | Rotavirus H |
| | | Rotavirus I |
| | | Rotavirus J |
| | Simplexvirus | Herpes simplex virus 1 |
| | | Herpes simplex virus 2 |
| | | Cercopihtecine herpesvirus |
| | Varicellovirus | Bovine alphaherpesvirus 1 |
| | | Bovine alphaherpesvirus 5 |
| | | Bubaline alphaherpesvirus 1 |
| | | Canid alphaherpesvirus 1 |
| | | Caprine alphaherpesvirus 1 |
| | | Cercopithecine alphaherpesvirus 9 |
| | | Cervid alphaherpesvirus 1 |
| | | Cervid alphaherpesvirus 2 |
| | | Cervid alphaherpesvirus 3 |
| | | Equid alphaherpesvirus 1 |
| | | Equid alphaherpesvirus 3 |
| | | Equid alphaherpesvirus 4 |
| | | Equid alphaherpesvirus 8 |
| | | Equid alphaherpesvirus 9 |
| | | Felid alphaherpesvirus 1 |
| | | Human alphaherpesvirus 3 |
| | | Monodontid alphaherpesvirus 1 |
| | | Phocid alphaherpesvirus 1 |
| | | Suid alphaherpesvirus 1 |
| | Lymphocryptovirus | Human gammaherpesvirus 4 (Epstein-Barr virus) |
| | Cytomegalovirus | Human betaherpesvirus 5 |
| | Rhadinovirus | Kaposi's sarcoma-associated herpesvirus |
| | | Murid gammaherpesvirus 4 |
| | Enterovirus | Enterovirus A (including subtype A71) |
| | | Enterovirus B (including coxsackie B virus subtypes) |
| | | Enterovirus C (including polioviruses 1, 2, and 3) |
| | | Rhinovirus A |
| | | Rhinovirus B |
| | | Rhinovirus C |
| | Alphainfluenzavirus | Influenza A |
| | Betainfluenzavirus | Influenza B |
| | Gammainfluenzavirus | Influenza C |
| | Deltainfluenzavirus | Influenza D |
| | Isavirus | Infections Salmon Anemia Virus |
| | Quaranjavirus | Johnston Atoll Virus |
| | | Quaranfil virus |
| | Thogotovirus | Dhori thogotovirus |
| | | Thogoto thogotovirus |
| | Morbilivirus | Measles morbilivirus |
| | | Canine morbillivirus (distemper) |
| | | Cetacean morbillivirus |
| | | Feline morbillivirus |
| | | Ovine rinderpest |
| | | Phocine morbillivirus |
| | | Rinderpest morbillivirus |
| | Rubivirus | Rubivirus rubellae |
| | | Rubivirus ruteetense |
| | | Rubivirus strelense |
| | Orthorubulavirus | Mumps orthorubulavirus |
| | Metapneumovirus | Avian metapneumovirus |
| | | Human metapneumovirus |
| | Orthopneumovirus | Human orthopneumovirus |
| | Alphapapillomavirus | Human alphapapillomavirus (all subtypes by L1 gene) |
| | Betapapillomavirus | Human betapapillomavirus (all subtypes by L1 gene) |
| | Gammapapillomavirus | Human gammapapillomavirus (all subtypes by L1 gene) |
| | Deltapapillomavirus | Bos taurus deltaapapillomavirus (all subtypes by L1 gene) |
| | Epsilonpapillomavirus | Bos taurus episilonpapillomavirus (all subtypes by L1 gene) |
| | Xipapillomavirus | Bos taurus Xipapillomavirus (all subtypes by L1 gene) |
| | Lentivirus | Human immunodeficiency virus 1 |
| | | Human immunodeficiency virus 2 |
| | Orthohantavirus | Andes orthohantavirus |
| | | Asama orthohantavirus |
| | | Asikkala orthohantavirus |
| | | Bayou orthohantavirus |
| | | Black Creek Canal orthohantavirus |
| | | Bowe orthohantavirus |
| | | Bruges orthohantavirus |
| | | Cano Delgadito orthohantavirus |
| | | Cao Bang orthohantavirus |
| | | Choclo orthohantavirus |
| | | Dabieshan orthohantavirus |
| | | Dobrava-Belgrade orthohantavirus |
| | | El Moro Canyon orthohantavirus |
| | | Fugong orthohantavirus |
| | | Fusong orthohantavirus |
| | | Hantaan orthohantavirus |
| | | Jeju orthohantavirus |
| | | Kenkeme orthohantavirus |
| | | Khabarovsk orthohantavirus |
| | | Laguna Negra orthohantavirus |
| | | Luxi orthohantavirus |
| | | Maporal orthohantavirus |
| | | Montano orthohantavirus |
| | | Necocli orthohantavirus |
| | | Oxbow orthohantavirus |
| | | Prospect Hill orthohantavirus |
| | | Puumala orthohantavirus |
| | | Robina orthohantavirus |
| | | Rockport orthohantavirus |
| | | Sangassou orthohantavirus |
| | | Seewis orhtohantavirus |
| | | Seoul orthohantavirus |
| | | Sin Nombre orthohantavirus |
| | | Tatenale orthohantavirus |
| | | Thailand orthohantavirus |
| | | Tigray orthohantavirus |
| | | Tula orthohantavirus |
| | | Yakeshi orthohantavirus |
| | Orthopoxvirus | Abatino macacapox virus |
| | | Akhmeta virus |
| | | Alaskapox virus |
| | | Camelpox virus |
| | | Cowpox virus |
| | | Ectromelia virus |
| | | Monkeypox virus |
| | | Raccoonpox virus |
| | | Skunkpox virus |
| | | Taterapox virus |
| | | Variola virus |
| | | Volepox virus |
| | Lyssavirus | Aravan lyssavirus |
| | | Australian bat lyssavirus |
| | | Bokeloh bat lyssavirus |
| | | Duvenhage lyssavirus |
| | | European bat 1 lyssavirus |
| | | European bat 2 lyssavirus |
| | | Gannoruwa bat lyssavirus |
| | | Irkut lyssavirus |
| | | Khujand lyssavirus |
| | | Madagascar bat lyssavirus |
| | | Rabies lyssavirus |
| | | Lagos bat lyssavirus |
| | | Mokola lyssavirus |
| | | Shimoni bat lyssavirus |
| | | West Caucasian bat lyssavirus |
| | | Ikoma lyssavirus |
| | | Lleida bat lyssavirus |
| | Mammarenavirus | Lymphocytic choriomeningitis virus |
| | Mastadenovirus | Human adenovirus A |
| | | Human adenovirus B |
| | | Human adenovirus C |
| | | Human adenovirus D |
| | | Human adenovirus E |
| | | Human adenovirus F |
| | | Human adenovirus G |

In some embodiments, the antigen comprises a viral polypeptide. In some embodiments, the viral polypeptide comprises a coronavirus polypeptide. In some embodiments, the coronavirus polypeptide comprises a SARS-CoV-2 polypeptide provided herein. In some embodiments, the SARS-CoV-2 polypeptide comprises a Spike protein or fragment thereof. In some embodiments, the Spike protein or fragment thereof is a full-length Spike protein. In some embodiments, the Spike protein or fragment thereof is a truncated Spike protein. In some embodiments, the truncated Spike protein comprises N-terminal domain of the Spike protein or S2 domain of the Spike protein. In some embodiments, the truncated Spike protein comprises N-terminal domain of the Spike protein and S2 domain of the Spike protein. In some embodiments, the Spike protein or fragment thereof is a recombinant Spike protein. In some embodiments, the Spike protein or fragment thereof comprises at least one modification. In some embodiments, the at least one modification comprises at least one amino acid mutation. In some embodiments, the at least one amino acid mutation eliminates a cleavage site such as a furin cleavage or serine protease cleavage site in the Spike protein. In some embodiments, the furin cleavage site comprises amino acid residues 682-685, amino acid residues 679-682, or amino acid residue 811 in SEQ ID NO: 21 (full-length Spike protein) or SEQ ID NO: 22 (Omicron mutant Spike protein).

In some embodiments, the serine protease cleavage site comprises amino acid residues 986 and 987; or 983 and 984 in SEQ ID NO: 21 (full-length Spike protein) or SEQ ID NO: 22 (Omicron mutant Spike protein). In some embodiments, the at least one amino acid mutation comprises amino acid substitution at amino acid residue 814, 889, 896, 939, 682-685, 679-682, 811, 986, 987, 983, 984, or a combination thereof in SEQ ID NO: 21 (full-length Spike protein) or SEQ ID NO: 22 (Omicron mutant Spike protein). In some embodiments, the at least one amino acid mutation comprises amino acid substitution at amino acid residue 814, 889, 896, and 939 in SEQ ID NO: 21 (full-length Spike protein) or SEQ ID NO: 22 (Omicron mutant Spike protein). In some embodiments, the Spike protein or fragment thereof comprises a signal peptide. In some embodiments, the signal peptide comprises an amino acid sequence of SEQ ID NO: 13: MDAMKRGLCCVLLLCGAVFVSASQEIHARFRR. In some embodiments, the signal peptide is a secretory peptide comprising an amino acid sequence that is at least 70%, at least 75%, at least 80%, at least 85%, at least 90%, at least 95%, or at least 99% identical to IgE Fc receptor alpha. In some embodiments, the secretory peptide is IgE Fc receptor alpha.

In some embodiments, the viral polypeptide comprises an influenza polypeptide. In some embodiments, the influenza polypeptide comprises an influenza A polypeptide, an influenza B polypeptide, an influenza C polypeptide, or an influenza D polypeptide. In some embodiments, the influenza polypeptide is the influenza A polypeptide. In some embodiments, the influenza A polypeptide comprises neuraminidase (NA) or fragment thereof or hemagglutinin (HA) or fragment thereof. In some embodiments, the influenza A polypeptide comprises the hemagglutinin (HA) or fragment thereof. In some embodiments, the hemagglutinin (HA) or fragment thereof is a full-length hemagglutinin (HA). In some embodiments, the hemagglutinin (HA) or fragment thereof is a truncated hemagglutinin (HA). In some embodiments, the truncated hemagglutinin (HA) comprises a Stalk domain. In some embodiments, the hemagglutinin (HA) or fragment thereof is a recombinant hemagglutinin (HA) comprising at least one modification. In some embodiments, the at least one modification comprises at least one amino acid mutation. In some embodiments, the at least one modification comprises the hemagglutinin (HA) or fragment thereof comprising an amino acid sequence of extracellular domain of M2 protein (M2e) of influenza A: SEQ ID NO: 14: MSLLTEVETPIRNEWGCRCNDSSD.

In some embodiments, the vector, e.g., recombinant retroviral vector, comprises a nucleic acid sequence encoding an enzyme. In some aspects, the enzyme can convert a nucleoside agent into a cytotoxic drug for killing a cell associated with the disease or condition described herein. In some embodiments, the enzyme comprises a kinase with nucleic acid nucleotide as a substrate. In some embodiments, the kinase is a thymidine kinase, where the thymidine kinase is salvage pathway enzyme which phosphorylates natural nucleoside substrates as well as nucleoside analogues. Generally, viral thymidine kinase can be exploited therapeutically by administration of a nucleoside analogue such as ganciclovir (GCV) or acyclovir to a cell expressing viral thymidine kinase, wherein the viral thymidine kinase phosphorylates the nucleoside analogue, creating a toxic product capable of killing the cell. Viral thymidine kinases of the present disclosure can be prepared from a wide variety of viral thymidine kinases. In some embodiments, the viral thymidine kinase mutant is derived from Herpesviridae thymidine kinase including, but not limited to, for example, both primate herpes viruses, and non-primate herpes viruses such as avian herpes viruses. Representative examples of suitable herpes viruses include, for example, Herpes Simplex Virus (HSV) Type 1, Herpes Simplex Virus Type 2, Varicella zoster Virus, marmoset herpes virus, feline herpes virus type 1, pseudorabies virus, equine herpes virus type 1, bovine herpes virus type 1, turkey herpes virus, Marek's disease virus, herpesvirus saimiri, or Epstein-Barr virus.

In some aspects, the thymidine kinase described herein can be a mutant thymidine kinase, where the mutant thymidine kinase comprises at least one amino acid mutation. In some aspects, the mutant thymidine kinase is a mutant Herpes Simplex Virus type 1 thymidine kinase (HSV1-TK) comprising at least one amino acid mutation compared to wild-type amino acid sequence of HSV1-TK: MASYPGHQHASAFDQAARSRGHSNRRTALRPRRQQEATEVRPEQKMPTLLRVYIDGPHGM GKTTTTQLLVALGSRDDIVYVPEPMTYWRVLGASETIANIYTTQHRLDQGEISAGDAAVVM TSAQITMGMPYAVTDAVLAPHIGGEAGSSHAPPPALTLIFDRHPIAALLCYPAARYLMGSMT PQAVLAFVALIPPTLPGTNIVLGALPEDRHIDRLAKRQRPGERLDLAMLAAIRRVYGLLANT VRYLQCGGSWREDWGQLSGTAVPPQGAEPQSNAGPRPHIGDTLFTLFRAPELLAPNGDLYN VFAWALDVLAKRLR (SEQ ID NO: 11). In some aspects, the mutant HSV1-TK comprises an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the HSV1-TK amino acid sequence (e.g., SEQ ID NO: 11). In some embodiments, the mutant HSV1-TK comprises a nuclear export sequence (NES). In some aspects, the NES comprises an amino acid sequence of LQKKLEELELDG (SEQ ID NO: 12).

Herpes viruses may be readily obtained from commercial sources such as the American Type Culture Collection (“ATCC”, Rockville, Md.). Herpes viruses may also be isolated from naturally occurring courses (e.g., an infected animal).

In some embodiments, the mutant HSV1-TK comprises at least one amino acid mutation at amino acid residue 25, 26, 32, 33, 167, 168, or a combination thereof compared to the wild-type amino acid sequence of HSV1-TK (SEQ ID NO: 11). In some embodiments, the mutation comprises substituting a wild-type amino acid with a polar, non-polar, basic or acidic amino acid. In some embodiments, the mutant HSV1-TK is mutated at amino acid residues 167, 168, or both. In one example, the sequence is mutated at amino acid residue 167. In another example, the sequence is mutated at amino acid residue 168. In another example, the sequence is mutated at amino acid residues 167 and 168. Amino acid residue 167 may be mutated to histidine, lysine, cysteine, serine, and phenylalanine. Amino acid residue 168 may be mutated to histidine, lysine, cysteine, serine, or phenylalanine. In some embodiments, the mutant HSV1-TK is mutated at amino acid residues 25 and/or 26. In amino acid residues 25 and/or 26 may be mutated to an amino acid chosen from the group consisting of: glycine, serine, and glutamate. In some embodiments, the mutant HSV1-TK is mutated at amino acid residues 32 and/or 33. Amino acid residues 32 and/or 33 may be mutated to an amino acid chosen from the group consisting of: glycine, serine, cysteine, glutamic acid, and aspartic acid. In some embodiments, the mutant HSV1-TK is mutated at amino acid residues 25, 26, 32, and/or 33. Amino acid residues 25, 26, 32, and/or 33, may be mutated to an amino acid chosen from the group consisting of: glycine, serine, cysteine, glutamic acid, and aspartic acid.

In some embodiments, the mutant HSV1-TK is mutated at amino acid residue: 25 and/or 26; and 167, where the mutation at amino acid residue 25 and/or 26 comprises glycine, serine, cysteine, glutamic acid, or aspartic acid; and the mutation at amino acid residue 167 comprises histidine, lysine, cysteine, serine, or phenylalanine. In some embodiments, the mutant HSV1-TK is mutated at amino acid residue: 25 and/or 26; and 168, where the mutation at amino acid residue 25 and/or 26 comprises glycine, serine, cysteine, glutamic acid, or aspartic acid; and the mutation at amino acid residue 168 comprises histidine, lysine, cysteine, serine, or phenylalanine. In some embodiments, the mutant HSV1-TK is mutated at amino acid residue: 25 and/or 26; and 167 and/or 168, where the mutation at amino acid residue 25 and/or 26 comprises glycine, serine, cysteine, glutamic acid, or aspartic acid; and the mutation at amino acid residue 167 and/or 168 comprises histidine, lysine, cysteine, serine, or phenylalanine.

In some embodiments, the mutant HSV1-TK is mutated at amino acid residue: 32 and/or 33; and 167, where the mutation at amino acid residue 32 and/or 33 comprises glycine, serine, cysteine, glutamic acid, or aspartic acid; and the mutation at amino acid residue 167 comprises histidine, lysine, cysteine, serine, or phenylalanine. In some embodiments, the mutant HSV1-TK is mutated at amino acid residue: 32 and/or 33; and 168, where the mutation at amino acid residue 32 and/or 33 comprises glycine, serine, cysteine, glutamic acid, or aspartic acid; and the mutation at amino acid residue 168 comprises histidine, lysine, cysteine, serine, or phenylalanine. In some embodiments, the mutant HSV1-TK is mutated at amino acid residue: 32 and/or 33; and 167 and/or 168, where the mutation at amino acid residue 32 and/or 33 comprises glycine, serine, cysteine, glutamic acid, or aspartic acid; and the mutation at amino acid residue 167 and/or 168 comprises histidine, lysine, cysteine, serine, or phenylalanine.

In some embodiments, the mutant HSV1-TK is mutated at amino acid residue: 25, 26, 32, and 33; and 167, where the mutation at amino acid residue 25, 26, 32, and 33 comprises glycine, serine, cysteine, glutamic acid, or aspartic acid; and the mutation at amino acid residue 167 comprises histidine, lysine, cysteine, serine, or phenylalanine. In some embodiments, the mutant HSV1-TK is mutated at amino acid residue: 25, 26, 32, and 33; and 168, where the mutation at amino acid residue 25, 26, 32, and 33 comprises glycine, serine, cysteine, glutamic acid, or aspartic acid; and the mutation at amino acid residue 168 comprises histidine, lysine, cysteine, serine, or phenylalanine. In some embodiments, the mutant HSV1-TK is mutated at amino acid residue: 25, 26, 32, and 33; and 167 and/or 168, where the mutation at amino acid residue 25, 26, 32, and 33 comprises glycine, serine, cysteine, glutamic acid, or aspartic acid; and the mutation at amino acid residue 167 and/or 168 comprises histidine, lysine, cysteine, serine, or phenylalanine.

In some embodiments, the mutant HSV1-TK is mutated at amino acid residue: 25 and 26 or 32 and 33; and 167, where the mutation at amino acid residue 25 and 26 or 32 and 33 comprises glycine, serine, cysteine, glutamic acid, or aspartic acid; and the mutation at amino acid residue 167 comprises histidine, lysine, cysteine, serine, or phenylalanine. In some embodiments, the mutant HSV1-TK is mutated at amino acid residue: 25 and 26 or 32 and 33; and 168, where the mutation at amino acid residue 25 and 26 or 32 and 33 comprises glycine, serine, cysteine, glutamic acid, or aspartic acid; and the mutation at amino acid residue 168 comprises histidine, lysine, cysteine, serine, or phenylalanine. In some embodiments, the mutant HSV1-TK is mutated at amino acid residue: 25 and 26 or 32 and 33; and 167 and/or 168, where the mutation at amino acid residue 25 and 26 or 32 and 33 comprises glycine, serine, cysteine, glutamic acid, or aspartic acid; and the mutation at amino acid residue 167 and/or 168 comprises histidine, lysine, cysteine, serine, or phenylalanine.

In some embodiments, the mutant HSV1-TK is mutated at amino acid residue: any one or more of 25, 26, 32 and/or 33; and 167, where the mutation at amino acid residue any one or more of 25, 26, 32 and/or 33 comprises glycine, serine, cysteine, glutamic acid, or aspartic acid; and the mutation at amino acid residue 167 comprises histidine, lysine, cysteine, serine, or phenylalanine. In some embodiments, the mutant HSV1-TK is mutated at amino acid residue: any one or more of 25, 26, 32 and/or 33; and 168, where the mutation at amino acid residue any one or more of 25, 26, 32 and/or 33 comprises glycine, serine, cysteine, glutamic acid, or aspartic acid; and the mutation at amino acid residue 168 comprises histidine, lysine, cysteine, serine, or phenylalanine. In some embodiments, the mutant HSV1-TK is mutated at amino acid residue: any one or more of 25, 26, 32 and/or 33; and 167 and/or 168, where the mutation at amino acid residue any one or more of 25, 26, 32 and/or 33 comprises glycine, serine, cysteine, glutamic acid, or aspartic acid; and the mutation at amino acid residue 167 and/or 168 comprises histidine, lysine, cysteine, serine, or phenylalanine.

In some embodiments, the vector, e.g., recombinant retroviral vector, in addition to comprising a nucleic acid sequence encoding mutant HSV1-TK, comprise a nucleic acid sequence encoding PiT-2, PiT-1, mCat-1 (murine cationic receptor-1; target of ecotropic Moloney MLV), or other receptors used by gamma retroviruses.

In some embodiments, the mutant HSV1-TK, compared to wild-type HSV1-TK (SEQ ID NO: 11), comprises increased enzymatic activity of converting a nucleoside agent into a cytotoxic drug. In some embodiments, the mutant HSV1-TK increases enzymatic activity of converting a nucleoside agent into a cytotoxic drug by at least 0.1 fold, 0.2 fold, 0.3 fold, 0.4 fold, 0.5 fold, 1.0 fold, 2.0 fold, 5.0 fold, 10.0 fold, or more compared to the enzymatic activity of a wild-type HSV1-TK converting the same nucleoside agent (e.g., a prodrug) into the cytotoxic drug.

In some embodiments, the mutant HSV1-TK increases bystander effect of cells capable of this phenomenon for killing the cell associated with the disease or condition. As used herein, the “bystander effect” refers to the phenomenon by which a HSV1-TK positive cell (e.g., cell contacted with vector described herein) exerts a kill effect on neighboring HSV1-TK negative cells following induction of HSV1-TK expression in the HSV1-TK positive cells. In some embodiments, the mutant HSV1-TK increases the bystander effect by at least 0.1 fold, 0.2 fold, 0.3 fold, 0.4 fold, 0.5 fold, 1.0 fold, 2.0 fold, 5.0 fold, 10.0 fold, or more compared to the bystander effect induced by a wild-type HSV1-TK positive cell.

### Vaccination

Described herein, in some embodiments, are vectors, e.g., a recombinant retroviral vectors, comprising a deleted integrase described herein. In some embodiments, the deleted integrase prevents the insertion of the vector genome into the genome of the host cell comprising the vector. In some embodiments, the vector, e.g., recombinant retroviral vector, comprises a mutant reverse transcriptase, wherein the mutant reverse transcriptase prevents converting of genetic materials of the vector into DNA for insertion into the genome of the target cell, e.g., host cell. In some embodiments, the vector, e.g., the recombinant retroviral vector, comprising the deleted integrase further comprises a mutant reverse transcriptase. In some embodiments, the vector, e.g., the recombinant retroviral vector, comprises both the deleted integrase and the mutant reverse transcriptase. Such vectors comprising a deleted integrase and/or mutant reverse transcriptase can be particularly useful and safe for vaccinating a subject in need thereof, where an antigen encoded by the vector can induce immune response of the subject without integrating genetic materials into genome of the host cell. The transient expression of the antigen can induce sufficient immune response against the antigen, thus vaccinating the subject against a disease or condition. In some embodiments, the expression of the antigen can be inducible, where the antigen is only expressed (thereby vaccinating the subject) when needed. For example, the expression of the antigen can be induced when the immunity against the antigen is waning, creating a booster vaccination.

In some cases, the antigen comprises a polypeptide sequence of a viral protein. In some embodiments, the antigen may be a viral protein of a coronavirus. In some embodiments, the coronavirus may be severe acute respiratory syndrome-related virus (SARS-CoV). In some embodiments, the SARS-CoV is SARS-CoV-2. In some embodiments, the epitope may be a viral protein selected from ORF1a, ORF1ab, Spike protein (S protein), 3a, 3b, envelope protein (E protein), matrix protein (M protein), p6, 7a, 7b, 8b, 9b, nucleocapsid protein (N protein), ORF14, Nsp1 (leader protein), Nsp2, Nsp3, Nsp4, Nsp5 (3C-like proteinase), Nsp6, Nsp7, Nsp8, Nsp9, Nsp10 (growth-factor-like protein), Nsp12 (RNA-dependent RNA polymerase, or RdRp), Nsp13 (RNA 5′-triphosphatase), Nsp14 (3′-to-5′ exonuclease), Nsp15 (endoRNAse), and Nsp16 (2′-O-ribose methyltransferase), a portion thereof, or combinations thereof.

In some embodiments, the antigen comprises a polypeptide sequence of a viral protein of an influenza virus. In some embodiments, the influenza virus is selected from the genera consisting of Influenza virus A, Influenza virus B, Influenza virus C and Influenza virus D. In further embodiments, the influenza A virus is of the subtype H1N1, H2N2, H3N2, H5N1, H7N7, H1N2, H9N2, H7N2, H7N3, H10N7, H7N9, or H6N1. In further embodiments, the influenza virus comprises influenza B virus of the B/Yamagata/16/88-like lineage or the B/Victoria/2/87-like lineage. In further embodiments, the influenza virus comprises influenza A virus of the A/California/04/2009-like lineage or the A/Brisbane/59/2007-like lineage. In some embodiments, the influenza may be any strain of the influenza virus or any serotypes within a stain of influenza virus. In some cases, the influenza virus comprises any combination viral surface glycoproteins haemagglutinin (H or HA) and neuraminidase (N or NA). In some embodiments, the antigen comprises modified influenza protein comprising hemagglutinin (HA) Stalk (conserved region) domain. In some embodiments, the HA stalk comprises at least one modification. Non-limiting examples of the HA stalk modification can include head removal; glycine linker loop; intra-Cys bridge; transmembrane removal; loop fusion peptide; GCN4 position; or inter-Cys bridge. In some embodiments, the antigen comprises Extracellular domain of the M2 protein (M2e) of influenza A has an amino acid sequence of: MSLLTEVETPIRNEWGCRCNDSSD (SEQ ID NO: 14).

In some embodiments, the antigen comprises a polypeptide sequence of a pathogen protein such as other viral protein, bacterial protein, parasite protein, fungus protein, or a combination thereof. In some embodiments, the antigen can include a tumor antigen such as Her2, where the subject is subsequently immunized against cancer.

### Method of Treatment

In some aspects, described herein are methods of treating a disease or condition in a subject by administering the vector, e.g., the recombinant retroviral vector described herein, to the subject, wherein the vector delivers at least one payload comprising a therapeutic or an antigen. In some embodiments, described herein are methods of treating a disease or condition in a subject by administering the vector, e.g., the recombinant retroviral vector described herein, to the subject, wherein the vector delivers at least one payload comprising a nucleic acid sequence, a nucleic acid molecule, a polypeptide or a fragment thereof, a cytokine, an enzyme, or a combination thereof. In some aspects, described herein is a method of vaccinating a subject by administering the vector, e.g., the recombinant retroviral vector described herein, to the subject, wherein the vector delivers an antigen as payload. In some instances, the antigen can then trigger innate immune response against the antigen, thus vaccinating the subject.

Disclosed herein, in some embodiments, are methods of using the vector, e.g., the recombinant retroviral vector, described herein. In some embodiments, the method comprises treating a disease or condition in a subject in need thereof by administering a vector, e.g., a recombinant retroviral vector, or pharmaceutical composition comprising the vector described herein to the subject. In some embodiments, the method comprises contacting a cell with the vector, e.g., the recombinant retroviral vector, and subsequently administering the cell to the subject. In some embodiments, the cell contacted with the vector, e.g., recombinant retroviral vector, is an autologous cell. For example, the cell can be first isolated from the subject and optionally cultured or expanded prior to being contacted with the vector. In some embodiments, expression of cytokines or HSV1-TK encoded by the vector, e.g., the recombinant retroviral vector, can be verified in the cell prior to administering the cell to the subject. In some embodiments, the cytokine comprises an interleukin (e.g., P40 or P35 of IL-12, IL-7) or an interferon.

In some embodiments, the method comprises administering two or more vectors, e.g., recombinant retroviral vectors, to the subject, where a first of the two or more vectors encode a cytokine described herein and a second of the two or more vectors encode a thymidine kinase (e.g., the mutated HSV1-TK) described herein. In some embodiments, the method comprises first contacting a cell with the two or more vectors, e.g., recombinant retroviral vectors, and subsequently administering the cell of the subject. In embodiments, the cytokine and the thymidine kinase (e.g., the mutated HSV1-TK) are encoded by the same vector, e.g., recombinant retroviral vector. In some embodiments, the cytokine comprises an interleukin (e.g., P40 or P35 of IL-12, IL-7) or an interferon. In some embodiments, administration is by any suitable mode of administration, including systemic administration (e.g., intravenous, inhalation, etc.). In some embodiments, the subject is human. In some embodiments, the disease or condition is cancer or lesion. In some embodiments, the disease or condition is metabolic disease. In some embodiments, the cancer comprises leukemia, myeloblastic, promyelocytic, myelomonocytic, monocytic, erythroleukemia, chronic myelocytic (granulocytic) leukemia, and chronic lymphocytic leukemia, lymphoma, e.g. Hodgkin's disease and non-Hodgkin's disease, fibrosarcoma, myosarcoma, liposarcoma, chondrosarcoma, osteogenic sarcoma, angio-sarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuro-blastoma, retinoblastoma, dysplasia and hyperplasia, prostate cancer, prostatitis, benign prostatic hypertrophy, benign prostatic hyperplasia (BPH), prostatic paraganglioma, prostate adenocarcinoma, prostatic intraepithelial neoplasia, rectal-prostate fistulas, and atypical prostatic stromal lesions. In some instances, the cancer comprises melanoma, epidermoid carcinoma, lung cancer, or glioblastoma.

In some embodiments, the disease or condition comprises a solid tumor. As used herein, in some instances, solid tumor refers to abnormal clump of cells or abnormal mass of tissue, which can be benign or malignant. In some instances, the solid tumor comprises sarcoma, carcinoma, or lymphoma.

In some embodiments, the vector, e.g., the recombinant retroviral vector, the cell comprising the vector, the recombinant virus comprising the vector, or the pharmaceutical composition comprising the vector, the cell, or the recombinant virus, deliver at least one payload, e.g., an interleukin, to a cell or microenvironment associated with the disease or condition. In some embodiments, the vector, e.g., the recombinant retroviral vector, the cell comprising the vector, the recombinant virus comprising the vector, or the pharmaceutical composition comprising the vector, the cell, or the recombinant virus, delivers at least one payload, e.g., an interleukin, and decreases toxicity (e.g., as determined by decreased cell death of cells not associated with the disease or condition or decreased expression of hot tumor genes such as CXCL9, CXCL10, and CXCL11) in the subject compared to direct administration of the at least one payload, e.g., the interleukin, to the subject. In some embodiments, the toxicity of delivering the at least one payload, e.g., the interleukin by the vector, e.g., the recombinant retroviral vector, the cell comprising the vector, the recombinant virus comprising the vector, or the pharmaceutical composition described herein is decreased by at least 0.1 fold, 0.2 fold, 0.5 fold, 1.0 fold, 2.0 fold, 5.0 fold, 10.0 fold, 50.0 fold, or more compared to toxicity induced by directly administering the at least one payload, e.g., the interleukin, to the subject.

In some embodiments, the vector, e.g., the recombinant retroviral vector, the cell comprising the vector, the recombinant virus comprising the vector, or the pharmaceutical composition delivers an IL-12 to a cell or microenvironment associated with the disease or condition. In some embodiments, the vector, e.g., the recombinant retroviral vector, the cell comprising the vector, the recombinant virus comprising the vector, or the pharmaceutical composition delivering the IL-12 (either as P40 subunit and P35 subunit or as a recombinant IL-12) decreases toxicity (e.g., as determined by decreased cell death of cells not associated with the disease or condition or decreased expression of hot tumor genes) in the subject compared to direct administration of IL-12 to the subject. In some embodiments, the toxicity of delivering the IL-12 by the vector, e.g., the recombinant retroviral vector, the cell comprising the vector, the recombinant virus comprising the vector, or the pharmaceutical composition described herein is decreased by at least 0.1 fold, 0.2 fold, 0.5 fold, 1.0 fold, 2.0 fold, 5.0 fold, 10.0 fold, 50.0 fold, or more compared to toxicity induced by directly administering the IL-12 to the subject.

In some embodiments, the IL-12 encoded by the vector, e.g., recombinant retroviral vector, is expressed or secreted by the cell. In some embodiments, the IL-12 expressed or secreted by the cell can stimulate innate immune signaling or response in the subject. In some embodiments, the method comprises stimulating the production of endogenous cytokines (e.g., IFN-γ) with the expressed or secreted interleukin (e.g., P40 or P35 of IL-12) for treating the disease or condition. Hot tumor gene expression can refer to expression of gene products such as the cytokines described herein that triggers endogenous immune response. As such, the expression of the hot tumor gene can lead to killing of the cell (e.g., a cancer or a tumor cell) associated with the disease or condition by the endogenous immune response.

In some embodiments, the vector, e.g., recombinant retroviral vector, the cell comprising the vector, the recombinant virus comprising the vector, or the pharmaceutical composition delivering the at least one payload, e.g., interleukin increases efficacy for treating the disease or condition (e.g., as determined by increased cell death of tumor cells or increased expression of hot tumor genes) in the subject compared to direct administration of the at least one payload, e.g., interleukin, to the subject. In some embodiments, the efficacy for treating the disease or condition by delivering the at least one payload, e.g., interleukin, by the vector, e.g., recombinant retroviral vector, the cell comprising the vector, the recombinant virus comprising the vector, or the pharmaceutical composition described herein is decreased by at least 0.1 fold, 0.2 fold, 0.5 fold, 1.0 fold, 2.0 fold, 5.0 fold, 10.0 fold, 50.0 fold, or more compared to efficacy for treating the disease or condition by directly administering the at least one payload, e.g., interleukin, to the subject.

In some embodiments, the vector, e.g., recombinant retroviral vector, the cell comprising the vector, the recombinant virus comprising the vector, non-viral genes comprising the vector, or the pharmaceutical composition delivering the IL-12 (either as P40 subunit and P35 subunit or as a recombinant IL-12) increases efficacy for treating the disease or condition (e.g., as determined by increased cell death of tumor cells or increased expression of hot tumor genes) in the subject compared to direct administration of IL-12 to the subject. In some embodiments, the efficacy for treating the disease or condition by delivering the IL-12 by the vector,, e.g., recombinant retroviral vector, the cell comprising the vector, the recombinant virus comprising the vector, or the pharmaceutical composition described herein is decreased by at least 0.1 fold, 0.2 fold, 0.5 fold, 1.0 fold, 2.0 fold, 5.0 fold, 10.0 fold, 50.0 fold, or more compared to efficacy for treating the disease or condition by directly administering the IL-12 to the subject.

In some embodiments, the vector, e.g., recombinant retroviral vector, the cell comprising the vector, the recombinant virus comprising the vector, or the pharmaceutical composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the pharmaceutical composition is administered two or more times (e.g., 2, 3, 4, 5, 6, 7, 8, 9, 10, 11, 12, 13, 14, 15, 16, 17, 18, 19, 20, 30, 40, 50, 60,70, 80, 90, 100 times) during a period of time.

Described herein, in some aspects, is a method of treating or preventing a disease or condition in a subject by vaccinating the subject, the method comprising administering to the subject a recombinant retroviral vector described herein, a recombinant virus comprising the recombinant retroviral vector described herein, the cell transduced by the vector, e.g., recombinant retroviral vector described herein, or the pharmaceutical composition described herein. In some embodiments, the at least one payload carried by the recombinant retroviral vector comprises an antigen that induces immune response in the subject, thereby treating or preventing the disease or condition in the subject by vaccinating the subject. In some embodiments, the immune response comprises induction of neutralizing antibody targeting the antigen, thereby generating immunity against the antigen in the subject. In some embodiments, the immune response comprises induction of immunoglobulin antibody targeting the antigen, thereby generating immunity against the antigen in the subject. In some embodiments, the immunoglobulin antibody comprises IgG antibody, IgM antibody, IgA antibody, IgD antibody, IgE antibody, or a combination thereof. In some embodiments, the immunoglobulin antibody comprises IgG antibody.

In some embodiments, the at least one payload is expressed in the subject for at least 12 hours, at least one day, at least two days, at least three days, at least four days, at least five days, at least six days, at least seven days, at least eight days, at least nine days, at least 10 days, at least 11 days, at least 12 days, or for a longer duration. In some embodiments, the at least one payload is expressed or secreted in the subject for at least 12 hours, at least one day, at least two days, at least three days, at least four days, at least five days, at least six days, at least seven days, at least eight days, at least nine days, at least 10 days, at least 11 days, at least 12 days, or for a longer duration.

In some embodiments, a duration of the immune response induced by the at least one payload expressed or secreted for at least 12 hours is increased by at least 10%, at least 20%, at least 50%, at least 100%, at least 5 fold, at least 10 fold, or more compared to a duration of an immune response induced by a comparable payload that is expressed or secreted for fewer than 12 hours. In some embodiments, a duration of the immune response induced by the at least one payload expressed or secreted for at least one day is increased by at least 10%, at least 20%, at least 50%, at least 100%, at least 5 fold, at least 10 fold, or more compared to a duration of an immune response induced by a comparable payload that is expressed or secreted for fewer than one day. In some embodiments, a duration of the immune response induced by the at least one payload expressed or secreted for at least two days is increased by at least 10%, at least 20%, at least 50%, at least 100%, at least 5 fold, at least 10 fold, or more compared to a duration of an immune response induced by a comparable payload that is expressed or secreted for fewer than two days. In some embodiments, a duration of the immune response induced by the at least one payload expressed or secreted for at least three days is increased by at least 10%, at least 20%, at least 50%, at least 100%, at least 5 fold, at least 10 fold, or more compared to a duration of an immune response induced by a comparable payload that is expressed or secreted for fewer than three days. In some embodiments, a duration of the immune response induced by the at least one payload expressed or secreted for at least four days is increased by at least 10%, at least 20%, at least 50%, at least 100%, at least 5 fold, at least 10 fold, or more compared to a duration of an immune response induced by a comparable payload that is expressed or secreted for fewer than four days. In some embodiments, a duration of the immune response induced by the at least one payload expressed or secreted for at least five days is increased by at least 10%, at least 20%, at least 50%, at least 100%, at least 5 fold, at least 10 fold, or more compared to a duration of an immune response induced by a comparable payload that is expressed or secreted for fewer than five days. In some embodiments, a duration of the immune response induced by the at least one payload expressed or secreted for at least five days is increased by at least 10%, at least 20%, at least 50%, at least 100%, at least 5 fold, at least 10 fold, or more compared to a duration of an immune response induced by a comparable payload that is expressed or secreted for fewer than six days. In some embodiments, a duration of the immune response induced by the at least one payload expressed or secreted for at least seven days is increased by at least 10%, at least 20%, at least 50%, at least 100%, at least 5 fold, at least 10 fold, or more compared to a duration of an immune response induced by a comparable payload that is expressed or secreted for fewer than seven days. In some embodiments, a duration of the immune response induced by the at least one payload expressed or secreted for at least eight days is increased by at least 10%, at least 20%, at least 50%, at least 100%, at least 5 fold, at least 10 fold, or more compared to a duration of an immune response induced by a comparable payload that is expressed or secreted for fewer than eight days. In some embodiments, a duration of the immune response induced by the at least one payload expressed or secreted for at least eight days is increased by at least 10%, at least 20%, at least 50%, at least 100%, at least 5 fold, at least 10 fold, or more compared to a duration of an immune response induced by a comparable payload that is expressed or secreted for fewer than nine days.

In some embodiments, the vector, e.g., recombinant retroviral vector, the cell comprising the vector, the recombinant virus comprising the vector, or the pharmaceutical composition is administered in a therapeutically-effective amount by various forms and routes including, but not limited to, oral, or topical administration. In some embodiments, the vector, e.g., recombinant retroviral vector, the cell comprising the vector, the recombinant virus comprising the vector, or the pharmaceutical composition may be administered by bronchial lavage, sublingual, intratumoral, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, or intraspinal administration, e.g., injection or infusion. In some embodiments, a pharmaceutical composition may be administered by absorption through epithelial or mucocutaneous linings (e.g., oral mucosa, rectal and intestinal mucosa administration). In some embodiments, the vector, e.g., recombinant retroviral vector, the cell comprising the vector, the recombinant virus comprising the vector, or the pharmaceutical composition is delivered via multiple administration routes.

In some embodiments, the vector, e.g., recombinant retroviral vector, the cell comprising the vector, the recombinant virus comprising the vector, or the pharmaceutical composition is administered by intravenous infusion. In some embodiments, the vector, e.g., recombinant retroviral vector, the cell comprising the vector, the recombinant virus comprising the vector, or the pharmaceutical composition comprising the vector is administered by slow continuous infusion over a long period, such as more than 24 hours. In some aspects, the vector, e.g., recombinant retroviral vector, the cell comprising the vector, the recombinant virus comprising the vector, or the pharmaceutical composition can be administered in a local manner, for example, via injection of the agent directly into an organ or a solid tumor, optionally in a depot or sustained release formulation or implant.

In some embodiments, the method comprises monitoring expression of the at least one payload, e.g., interleukin such as IL-12, in the subject after the subject has been treated. In some aspects, the method comprises monitoring the expression level of the at least one payload, e.g., IL-12, wherein, if the payload, e.g., IL-12, expression reaches a predetermined threshold in the subject, an additional therapeutic, e.g., interleukin inhibitor, can be administered to the subject.

In some embodiments, the vector, e.g., recombinant retroviral vector, the cell comprising the vector, the recombinant virus comprising the vector, or the pharmaceutical composition provided herein can be administered in conjunction with at least one additional therapeutic, for example, an antiviral therapy, a chemotherapy, an antibiotic, a cell therapy, a cytokine therapy, or an anti-inflammatory agent. In some embodiments, the at least one additional therapeutic comprises a nucleoside agent (e.g., a prodrug). Non-limiting example of the prodrug can include FHBG (9-[4-fluoro-3-(hydroxymethyl)butyl]guanine), FHPG (9-([3-fluoro-1-hydroxy-2-propoxy]methyl)guanine), FGCV (fluoroganciclovir), FPCV (fluoropenciclovir), FIAU (1-(2′-deoxy-2′-fluoro-1-β-D-arabinofuranosyl)-5-iodouracil), FEAU (fluoro-5-ethyl-1-beta-D-arabinofuranosyluracil), FMAU (fluoro-5-methyl-1-beta-D-arabinofuranosyluracil), FHOMP (6-((1-fluoro-3-hydroxypropan-2-yloxy)methyl)-5-methylpryrimidine-2,4(1H,3H)-dione), ganciclovir, valganciclovir, acyclovir, valacivlovir, penciclovir, radiolabeled pyrimidine with 4-hydroxy-3-(hydroxymethyl)butyl side chain at N-1 (HHG-5-FEP) or 5-(2-)hydroxyethyl)- and 5-(3-hydroxypropyl)-substituted pyrimidine derivatives bearing 2,3-dihydroxypropyl, acyclovir-, ganciclovir-and penciclovir-like side chains for thymidine kinase; ifosfamide for oxidoreductase; 6-methoxypurine arabinoside for VZV-TK; 5-fluorocytosine for cytosine deaminase; doxorubicin for beta-glucuronidase; CB1954 for nitroreductase; and N-(Cyanoacetyl)-L-phenylalanine, or N-(3-chloropropionyl)-L-phenylalanine for carboxypeptidase A. In some embodiments, the nucleoside agent comprises ganciclovir, valganciclovir, acyclovir, valacyclovir, or penciclovir.

In some embodiments, the vector, e.g., recombinant retroviral vector, the cell comprising the vector, the recombinant virus comprising the vector, or the pharmaceutical composition provided herein can be administered before, during, or after occurrence of the disease or condition. In some embodiments, the vector, e.g., recombinant retroviral vector, the cell comprising the vector, the recombinant virus comprising the vector, or the pharmaceutical composition can be used as a prophylactic and may be administered continuously to subjects. In some embodiments, the vector, e.g., recombinant retroviral vector, the cell comprising the vector, the recombinant virus comprising the vector, or the pharmaceutical composition can be administered to a subject before the onset of the symptoms associated with the disease or condition.

Actual dosage levels of an agent of the disclosure (e.g., the vector, e.g., recombinant retroviral vector, the cell comprising the vector, the recombinant virus comprising the vector, or the pharmaceutical composition) can be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors such as the activity of the agent of the present disclosure (e.g., the vector, e.g., recombinant retroviral vector, the cell comprising the vector, the recombinant virus comprising the vector, or the pharmaceutical composition), the route of administration, the time of administration, the rate of excretion, the duration of the treatment, other drugs, compounds and/or materials used in combination with the agent, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear biomarkers, e.g., polyribonucleotide or antibody or antigen-binding fragment thereof.

In some embodiments, the vector, e.g., recombinant retroviral vector, the cell comprising the vector, the recombinant virus comprising the vector, or the pharmaceutical composition described herein can be in a unit dosage form suitable for a single administration of a precise dosage. In unit dosage form, the formulation may be divided into unit doses containing appropriate quantities of the vector, e.g., recombinant retroviral vector, the cell comprising the vector, the recombinant virus comprising the vector, or the pharmaceutical composition described herein. In unit dosage form, the formulation may be divided into unit doses containing appropriate quantities of one or more biomarkers, e.g., linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

The dosage of the vector, e.g., recombinant retroviral vector, the cell comprising the vector, the recombinant virus comprising the vector, or the pharmaceutical composition lies preferably within a range of circulating concentrations that include the ED50 with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC50 (i.e., the concentration of the test compound which achieves a half-maximal infection or a half-maximal inhibition) as determined in cell culture or other suitable assays. Such information can be used to determine useful doses more accurately in humans. Levels in plasma may be measured, for example, by RT-qPCR or ddPCR methods.

An effective amount or therapeutically effective amount of the vector, e.g., recombinant retroviral vector, the cell comprising the vector, the recombinant virus comprising the vector, or the pharmaceutical composition disclosed herein to be administered to a subject in need of treatment may be determined in a variety of ways. By way of example, the amount may be based on viral titer or efficacy in an in vitro, or an animal model. Alternatively, the dosing regimens used in clinical trials may be used as general guidelines.

In some embodiments, the daily dose may be administered in a single dose or in portions at various hours of the day. In some embodiments, a higher dosage may be required and may be reduced over time when the optimal initial response is obtained. In some embodiments, treatment may be continuous for days, weeks, or years, or may be at intervals with intervening rest periods. In some embodiments, the dosage is modified in accordance with other treatments the individual may be receiving. However, the method of treatment is in no way limited to a particular concentration or range of the vector, e.g., recombinant retroviral vector, the cell comprising the vector, the recombinant virus comprising the vector, or the pharmaceutical composition described herein and can be varied for each individual being treated and for each agent used. Individualization of dosage may be required to achieve the maximum effect for a given individual. In some embodiments, the dosage administered to an individual being treated varies depending on the individual's age, severity or stage of the disease and response to the course of treatment. In some embodiments, clinical parameters for determining dosage include, but are not limited to, tumor size, alteration in the level of tumor markers used in clinical testing for particular malignancies. In some embodiments, the treating physician determines the therapeutically effective amount to be used for a given individual. In some embodiments, the therapies disclosed herein are administered as often as necessary and for the period of time judged necessary by the treating physician.

In some embodiments, multiple therapeutic courses (e.g., first and second therapeutic course) are administered to a subject in need of treatment. In some embodiments, the first and/or second therapeutic course is administered intravenously. In other embodiments, the first and/or second therapeutic course is administered via intra-arterial infusion, including but not limited to infusion through the hepatic artery, cerebral artery, coronary artery, pulmonary artery, iliac artery, celiac trunk, gastric artery, splenic artery, renal artery, gonadal artery, subclavian artery, vertebral artery, axillary artery, brachial artery, radial artery, ulnar artery, carotid artery, femoral artery, inferior mesenteric artery and/or superior mesenteric artery. Intra-arterial infusion may be accomplished using endovascular procedures, percutaneous procedures or open surgical approaches. In some embodiments, the first and second therapeutic course may be administered sequentially. In yet other embodiments, the first and second therapeutic course may be administered simultaneously. In still other embodiments, the optional third therapeutic course may be administered sequentially or simultaneously with the first and second therapeutic courses.

In some embodiments, the vector, e.g., recombinant retroviral vector, the cell comprising the vector, the recombinant virus comprising the vector, or the pharmaceutical composition disclosed herein may be administered in conjunction with a sequential or concurrently administered therapeutic course(s) in high doses on a cumulative basis. For example, in some embodiments, a patient in need thereof may be systemically administered, e.g., intravenously administered, with a therapeutic course on a cumulative basis. A first therapeutic course may be systemically administered. Alternatively, the first therapeutic course may be administered in a localized manner, e.g., intra-arterially, for example a patient in need thereof may be administered via intra-arterial infusion on a cumulative basis.

In yet other embodiments, a subject in need thereof may receive a combination, either sequentially or concurrently, of systemic and intra-arterial infusions administration of high doses of the vector, e.g., recombinant retroviral vector, the cell comprising the vector, the recombinant virus comprising the vector, or the pharmaceutical composition. For example, a patient or a subject in need thereof may be first systemically administered with the vector, e.g., recombinant retroviral vector, the cell comprising the vector, the recombinant virus comprising the vector, or the pharmaceutical composition on a cumulative basis, followed by an additional therapeutic course of intra-arterial infusion, e.g., hepatic arterial infusion, administered delivery on a cumulative basis.

A subject in need of treatment may also be administered, either systemically or localized (for example intra-arterial infusion, such as hepatic arterial infusion) a therapeutic course of delivering the vector, e.g., recombinant retroviral vector, the cell comprising the vector, the recombinant virus comprising the vector, or the pharmaceutical composition for a defined period of time. In some embodiments, the period of time may be at least one day, at least two days, at least three days, at least four days, at least five days, at least six days, at least seven days, at least one week, at least two weeks, at least three weeks, at least four weeks, at least five weeks, at least six weeks, at least seven weeks, at least eight weeks, at least 2 months, at least three months, at least four months, at least five months, at least six months, at least seven months, at least eight months, at least nine months, at least ten months, at least eleven months, at least one year, at least two years, at least three years, at least four years, or at least five years. Administration could also take place in a chronic manner, i.e., for an undefined or indefinite period of time.

Administration of the vector, e.g., recombinant retroviral vector, the cell comprising the vector, the recombinant virus comprising the vector, or the pharmaceutical composition may also occur in a periodic manner, e.g., at least once a day, at least twice a day, at least three times a day, at least four times a day, at least five times a day. Periodic administration of the delivery of the vector, e.g., recombinant retroviral vector, the cell comprising the vector, the recombinant virus comprising the vector, or the pharmaceutical composition may be dependent upon the time of delivery as well as the mode of administration. For example, parenteral administration may take place only once a day over an extended period of time, whereas oral administration of the delivery of the vector, e.g., recombinant retroviral vector, the cell comprising the vector, the recombinant virus comprising the vector, or the pharmaceutical composition may take place more than once a day wherein administration of the delivery of the vector, e.g., recombinant retroviral vector, the cell comprising the vector, the recombinant virus comprising the vector, or the pharmaceutical composition takes place over a shorter period of time.

In one embodiment, the subject is allowed to rest 1 to 2 days between the first therapeutic course and second therapeutic course. In some embodiments, the subject is allowed to rest 2 to 4 days between the first therapeutic course and second therapeutic course. In other embodiments, the subject is allowed to rest at least 2 days between the first and second therapeutic course. In yet other embodiments, the subject is allowed to rest at least 4 days between the first and second therapeutic course. In still other embodiments, the subject is allowed to rest at least 6 days between the first and second therapeutic course. In some embodiments, the subject is allowed to rest at least 1 week between the first and second therapeutic course. In yet other embodiments, the subject is allowed to rest at least 2 weeks between the first and second therapeutic course. In one embodiment, the subject is allowed to rest at least one month between the first and second therapeutic course. In some embodiments, the subject is allowed to rest at least 1-7 days between the second therapeutic course and the optional third therapeutic course. In yet other embodiments, the subject is allowed to rest at least 1-2 weeks between the second therapeutic course and the optional third therapeutic course.

In some embodiments, the vector, e.g., recombinant retroviral vector, the cell comprising the vector, the recombinant virus comprising the vector, or the pharmaceutical composition is administered to increase local concentration of an interleukin (e.g., P40 or P35 of IL-12) and a thymidine kinase (e.g., the mutated HSV1-TK) in the cell or the microenvironment associated with the disease or condition (e.g., cancer or lesion) described herein. In some embodiments, the vector, e.g., recombinant retroviral vector, the cell comprising the vector, the recombinant virus comprising the vector, or the pharmaceutical composition is administered via intra-arterial infusion, which increases local concentration of the therapeutic vector to a specific organ system. In yet other embodiments, the vector, e.g., recombinant retroviral vector, the cell comprising the vector, the recombinant virus comprising the vector, or the pharmaceutical composition is administered intratumorally. Dependent upon the location of the target lesions, in some embodiments, catheterization of the hepatic artery is followed by infusion into the pancreaticoduodenal, right hepatic, and middle hepatic artery, respectively, in order to locally target hepatic lesions. In some embodiments, localized distribution to other organ systems, including, but not limited to, the lung, gastrointestinal, brain, reproductive, splenic or other defined organ system, of the polypeptide or delivery vector is accomplished via catheterization or other localized delivery system. In some embodiments, intra-arterial infusions are accomplished via any other available arterial source, including but not limited to infusion through the hepatic artery, cerebral artery, coronary artery, pulmonary artery, iliac artery, celiac trunk, gastric artery, splenic artery, renal artery, gonadal artery, subclavian artery, vertebral artery, axillary artery, brachial artery, radial artery, ulnar artery, carotid artery, femoral artery, inferior mesenteric artery and/or superior mesenteric artery. In some embodiments, intra-arterial infusion is accomplished using endovascular procedures, percutaneous procedures or open surgical approaches.

### Pharmaceutical Composition

Described herein is a pharmaceutical composition comprising a therapeutic agent (e.g., the vector e.g., recombinant retroviral vector, or the cell comprising the vector described herein) or an antigen for vaccinating a subject. In some aspects, the cell contacted with the vector e.g., recombinant retroviral vector, described herein expresses at least one payload, e.g., an interleukin (e.g., P40 or P35 of IL-12), a thymidine kinase (e.g., the mutated HSV1-TK), or an antigen described herein in vivo or in vitro. In some aspects, the cell is: obtained from a subject; expanded in an in vitro environment; and administered back to the subject for treating the disease or condition in the subject or vaccinating the subject. In some embodiments, the vector e.g., recombinant retroviral vector, or cell contacted with the vector described herein can be formulated into a vaccine. In some embodiments, the vector e.g., recombinant retroviral vector, described herein is formulated into an RNA vaccine. In some embodiments, the vector e.g., recombinant retroviral vector, described herein is formulated into a mRNA vaccine, where the antigen is encoded by mRNA as the payload of the vector. In some embodiments, the pharmaceutical composition comprises a recombinant virus comprising a vector, e.g., recombinant retroviral vector, described herein. In some instances, the pharmaceutical composition comprises a pseudotyped viral vector comprising a modified Sindbis virus envelope protein, for example a modified Sindbis virus envelope protein targeting dendritic cells. In some embodiments, the vaccine comprises at least two vectors, e.g., recombinant retroviral vector, described herein, wherein the at least two vectors can encode different payloads. In some embodiments, the vector, e.g., recombinant retroviral vector, comprises the deleted integrase described herein, wherein the viral genome can no longer be inserted into the genome of the host cell. In some embodiments, the vector, e.g., recombinant retroviral vector, comprises the mutant reverse transcriptase, where the genetic materials, e.g., nucleic acid (e.g., RNA) of the vector can no longer be converted into DNA and subsequently inserted into the genome of the cell. In some embodiments, the vector, e.g., recombinant retroviral vector, comprises both the deleted integrase and the mutant reverse transcriptase.

In some aspects, the cell is obtained from a source that is not from the subject. In some aspect, the cell is obtained from a cell line. In some embodiments, the cell is formulated into the pharmaceutical composition. In some embodiments, the pharmaceutical composition comprises a nucleoside agent described herein.

In some aspects, the pharmaceutical composition further comprises a pharmaceutically acceptable: carrier, excipient, or diluent. In some aspects, the pharmaceutical composition described herein comprises at least one additional active agent other than the cell described herein. In some aspects, the at least one additional active agent is a chemotherapeutic agent, cytotoxic agent, cytokine, growth-inhibitory agent, anti-hormonal agent, anti-angiogenic agent, or checkpoint inhibitor.

In some aspects, the pharmaceutical composition comprises an adjuvant for augmenting immune response for vaccinating the subject in need thereof. In some embodiments, the adjuvant can comprise analgesic adjuvants. In some embodiments, the adjuvant can comprise inorganic compounds such as alum, aluminum hydroxide, aluminum phosphate, or calcium phosphate hydroxide. In some embodiments, the adjuvant can comprise mineral oil or paraffin oil. In some embodiments, the adjuvant can comprise bacterial products such as inactivated Bordetella pertussis, Mycobacterium bovis, or oxoids. In some embodiments, the adjuvant can comprise nonbacterial organics like squalene. In some embodiments, the adjuvant can comprise the use of delivery systems such as detergents (Quil A). In some embodiments, the adjuvant can comprise plant saponins such as saponin derived from Quillaja, soybean, or Polygala senega. In some embodiments, the adjuvant can comprise Freund's complete adjuvant or Freund's incomplete adjuvant. In some embodiments, the adjuvant can comprise food-based oil like peanut oil.

In practicing the methods of treatment or use provided herein, therapeutically effective amount of pharmaceutical composition described herein is administered to a subject, e.g., a mammal having a disease or condition to be treated, e.g., cancer or lesion. In some aspects, the subject is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to bronchial lavage, sublingual, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some embodiments, the pharmaceutical composition is administered to a subject systemically. In some embodiments, the pharmaceutical composition is administered intravenously, intraarterially, orally, subcutaneously, intramuscularly, or intraperitoneally. In some embodiments, the pharmaceutical composition is administered intravenously or intraarterially.

The pharmaceutical composition comprising a therapeutic agent (e.g., the vector e.g., recombinant retroviral vector, or the cell comprising the vector described herein) can be manufactured in a conventional manner, such as, by way of example only, by means of conventional mixing, dissolving, granulating, dragee-making, levigating, emulsifying, encapsulating, entrapping or compression processes.

The pharmaceutical compositions may include, but are not limited to, at least an exogenous therapeutic agent as an active ingredient in free-acid or free-base form, or in a pharmaceutically acceptable salt form. In addition, the methods and compositions described herein include, but are not limited to, the use of N-oxides (if appropriate), crystalline forms, amorphous phases, as well as active metabolites of these compounds having the same type of activity. In some aspects, therapeutic agents exist in unsolvated form or in solvated forms with pharmaceutically acceptable solvents such as water, ethanol, and the like. The solvated forms of the therapeutic agents are also considered to be disclosed herein.

In certain embodiments, the pharmaceutical composition provided herein includes, but is not limited to, one or more preservatives to inhibit microbial activity. Suitable preservatives include, but are not limited to, mercury-containing substances such as phenylmercuric borate and thiomersal; stabilized chlorine dioxide; and quaternary ammonium compounds such as benzalkonium chloride, cetyltrimethylammonium bromide and cetylpyridinium chloride.

In some aspects, pharmaceutical composition described herein benefits from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical composition described herein can be formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some aspects, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a pharmaceutical composition described herein is formulated in aqueous solutions, such as in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include, but are not limited to, aqueous or nonaqueous solutions, such as with physiologically compatible buffers or excipients.

Parenteral injections may involve bolus injection or continuous infusion. Pharmaceutical composition for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations comprising a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

In another aspect, dosage forms include, but are not limited to, microencapsulated formulations. In some aspects, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some aspects, the aqueous dispersions further include, but are not limited to, a crystal-forming inhibitor.

In some aspects, the pharmaceutical composition described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some aspects, SEDDS provides improvements in the bioavailability of hydrophobic active ingredients.

Buccal formulations are administered using a variety of formulations known in the art. In addition, the buccal dosage forms described herein may further include, but are not limited to, a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical composition is optionally formulated in aqueous solutions, such as in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include, but are not limited to, aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some aspects, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include, but are not limited to, aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, but are not limited to, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., Wurster coating), tangential coating, top spraying, tableting, extruding and the like.

In some aspects, the pharmaceutical composition comprising a therapeutic agent (e.g., the vector e.g., recombinant retroviral vector, or the cell comprising the vector described herein) and at least one dispersing agent or suspending agent can be formulated for oral administration to a subject. The formulations may be a powder and/or granule for suspension, and upon admixture with water, a substantially uniform suspension is obtained.

In some aspects, the pharmaceutical composition comprises an agent that facilitates vector binding and/or entry into a cell for either in vivo or ex vivo application. Certain pharmaceutical compositions comprise a poly-cationic agent, either for co-administration or for pre-formulation with the vector described herein prior to administration to a patient, a subject, or to a cell derived from a patient or a subject. Such poly-cationic agents may include, but are not limited to, polybrene, protamine sulfate, or recombinant human fibronectin.

Furthermore, in some cases the pharmaceutical composition comprises one or more pH adjusting agents or buffering agents, including, but not limited to, acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, in some cases the pharmaceutical composition comprises one or more salts in an amount required to bring osmolality of the pharmaceutical composition into an acceptable range. Such salts include, but are not limited to, those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration, in some cases, comprises an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including, but not limited to, halocarbons, for example, fluorocarbons such as fluorinated chlorinated hydrocarbons, hydrochlorofluorocarbons, and hydrochlorocarbons, as well as hydrocarbons and hydrocarbon ethers. Aerosol formulations, in some cases, comprise other components, for example, ethanol, isopropanol, propylene glycol, as well as surfactants or other components such as oils and detergents. These components may serve to stabilize the formulation and/or lubricate valve components.

### Kit

Described herein, in some aspects, are kits for using the vector, e.g., recombinant retroviral vector, described herein. In some embodiments, the kit can be used to treat a disease or condition in a subject. In some embodiments, the kit can be used to vaccinate a subject. In some aspects, the kit comprises an assemblage of materials or components apart from the vector, e.g., recombinant retroviral vector, or a cell comprising the vector. In some aspects, the kit comprises the components for assaying the number of units of a biomolecule (e.g., a therapeutic agent comprising the vector, e.g., recombinant retroviral vector, the cell, IL-12, the mutant HSV1-TK, the antigen such as Spike protein or HA protein or a combination thereof) synthesized, and/or released or expressed by the cell described herein. In some aspects, the kit comprises components for performing assays such as enzyme-linked immunosorbent assay (ELISA), single-molecular array (Simoa), PCR, and qPCR. The exact nature of the components configured in the kit depends on its intended purpose. For example, kits can be configured for the purpose of treating a disease or condition disclosed herein (e.g., cancer or lesion) in a subject. In some aspects, the kit is configured particularly for the purpose of treating mammalian subjects. In some aspects, the kit is configured particularly for the purpose of treating human subjects. In some aspects, the kit is configured particularly for the purpose of vaccinating mammalian subjects. In some aspects, the kit is configured particularly for the purpose of vaccinating human subjects.

In some instances, the kit comprises instructions. In some aspects, the kit comprises instructions for administering the vector, e.g., recombinant retroviral vector, the cell, or the pharmaceutical composition described herein to a subject in need thereof. In some aspects, the kit comprises instructions for further engineering the vector, e.g., recombinant retroviral vector, or cell to express a payload or a biomolecule (e.g., a therapeutic agent such as the IL-12, HSV1-TK, or the antigen). In some aspects, the kit comprises instructions thawing or otherwise restoring biological activity of the cell, which may have been preserved during storage or transportation. In some aspects, the kit comprises instructions for measuring viability of the preserved cell, to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are often contained in suitable packaging material.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein, “nucleic acid” refers to a polynucleotide containing at least two covalently linked nucleotide or nucleotide analog subunits. A nucleic acid is generally a deoxyribonucleic acid (DNA), a ribonucleic acid (RNA), or an analog of DNA or RNA. The nucleic acid is generally single-stranded, double-stranded, or a mixture thereof. For purposes herein, unless specified otherwise, the nucleic acid is double-stranded, or it is apparent from the context.

As used herein, “DNA” is meant to include all types and sizes of DNA molecules including cDNA, plasmids and DNA including modified nucleotides and nucleotide analogs.

As used herein, “nucleotides” include nucleoside mono-, di-, and triphosphates. Nucleotides also include modified nucleotides, such as, but are not limited to, phosphorothioate nucleotides and deazapurine nucleotides and other nucleotide analogs.

The term “polynucleotide” as used herein means a polymeric form of nucleotide of any length, and includes ribonucleotides and deoxyribonucleotides. Such term also includes single-and double-stranded DNA, as well as single-and double-stranded RNA. The term also includes modified polynucleotides such as methylated or capped polynucleotides.

As used herein, the term “subject” refers to animals, plants, insects, and birds into which the large DNA molecules are introduced. Included are higher organisms, such as mammals and birds, including humans, primates, rodents, cattle, pigs, rabbits, goats, sheep, mice, rats, guinea pigs, cats, dogs, horses, chicken and others. Subject may or may not have a disease or condition.

As used herein, “administering to a subject” is a procedure by which one or more delivery agents and/or vectors, e.g., recombinant retroviral vector described herein, together or separately, are introduced into or applied onto a subject such that target cells which are present in the subject are eventually contacted with the agent and/or vector, e.g., recombinant retroviral vector.

As used herein, “delivery vector” or “delivery vehicle” or “therapeutic vector” or “therapeutic system” refers to both viral and non-viral particles that harbor and transport exogenous nucleic acid molecules to a target cell or tissue. Viral vehicles include, but are not limited to, retroviruses, adenoviruses, lentiviruses, herpes viruses and adeno-associated viruses. Non-viral vehicles include, but are not limited to, microparticles, nanoparticles, virosomes and liposomes. “Targeted,” as used herein, refers to the use of ligands that are associated with the delivery vehicle and target the vehicle to a cell or tissue. Ligands include, but are not limited to, antibodies or fragments thereof, diabodies, receptors, and collagen-binding domains.

As used herein, “delivery,” which is used interchangeably with “transduction,” refers to the process by which exogenous nucleic acid molecules are transferred into a cell such that they are located inside the cell. Delivery of nucleic acids is a distinct process from expression of nucleic acids.

As used herein, “expression” refers to the process by which nucleic acid is translated into polypeptide or is transcribed into RNA, which, for example, can be translated into polypeptide or protein. If the nucleic acid is derived from genomic DNA, expression includes, if an appropriate eukaryotic host cell or organism is selected, splicing of the mRNA. For heterologous nucleic acid to be expressed in a host cell, it must initially be delivered into the cell and then, once in the cell, ultimately reside in the nucleus. In some aspects, the expression occurs independent of gene integration.

As used herein, a “therapeutic course” refers to the periodic or timed administration of the vectors disclosed herein within a defined period of time. Such a period of time is at least one day, at least two days, at least three days, at least five days, at least one week, at least two weeks, at least three weeks, at least one month, at least two months, or at least six months. Administration could also take place in a chronic manner, i.e., for an undefined period of time. The periodic or timed administration includes once a day, twice a day, three times a day or other set timed administration.

As used herein, the terms “co-administration,” “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments, a therapeutic agent as disclosed in the present application will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, a therapeutic agent and the other agent(s) are administered in a single composition. In some embodiments, a therapeutic agent and the other agent(s) are admixed in the composition. In further embodiments, a therapeutic agent and the other agent(s) are administered at separate times in separate doses.

As used herein, the term “mutant thymidine kinase” refers to not only the specific protein described herein (as well as the nucleic acid sequences which encode these proteins), but derivatives thereof which may include various structural forms of the primary protein which retain biological activity.

As used herein, the term “mutated” or “replaced by another nucleotide” means a nucleotide at a certain position is replaced at that position by a nucleotide other than that which occurs in the unmutated or previously mutated sequence. That is, in some instances, specific modifications may be made in different nucleotides. In some embodiments, the replacements are made such that the relevant splice donor and/or acceptor sites are no longer present in a gene.

As used herein, the term “deleted” means a stretch of nucleotides or a nucleic acid sequence at a certain position has been removed. In some cases, a portion of nucleotides or a nucleic acid sequence encoding a gene are removed or deleted. In some cases, all of the nucleotides or nucleic acid sequence encoding a gene are removed or deleted.

As used herein, a “polar amino acid” refers to amino acid residues Asn (N), Cys (C), Gln (Q), Gly (G), Ser(S), Thr (T) or Tyr (Y).

As used herein, a “non-polar amino acid” refers to amino acid residues Ala (A), Ile (I), Leu (L), Met (M), Phe (F), Pro (P), Trp (W), or Val (V).

As used herein, a “basic amino acid” refers to amino acid residues Arg (R), His (H), or Lys (K).

As used herein, an “acidic amino acid” refers to amino acid residues Asp (D) or Glu (E).

An “adjuvant” as described herein refers to a substance that in combination with an antigen promotes an adaptive immune response to the antigen. An “immune stimulatory compound” refers to a substance that specifically interacts with the innate immune system to initiate a “danger signal” that ultimately leads to the development of the adaptive components of the immune response (e.g., B cell, T cells). Immune stimulatory compounds include pathogen-associated molecular patterns (PAMPs) such as dsRNA, lipopolysaccharide, and CpG DNA, either naturally occurring or synthetic. Immune stimulatory compounds are agonists of various innate immune receptors including Toll-like receptors (TLRs), NOD-like receptors, RIG-1 or MDA-5 receptors, C-type lectin receptors, or the STING pathway.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Integrase Deleted Gag-Pol Viral Vectors

A modified murine leukemia virus (MLV) Gag-Pol was produced to completely eliminate the integrase from the MLV Gag-Pol polyprotein (FIG. 1). This was achieved via site-directed mutagenesis using GenVivo's GP340-VKS plasmid vector as starting material. Primers (FWD-5′-TAATGAGCGGCCGCGAAG-3′ (SEQ ID NO: 24); REV-5′-GAGGAGGGTAGAGGTGTC-3′ (SEQ ID NO: 25)) were used to amplify from GP340-VKS with Q5 Hot Start High-Fidelity Polymerase (New England Biolabs). The primers were designed to flank the integrase such that resulting amplicon would contain truncated gagpol, excluding the integrase but retaining the original TAA stop codon from the 3′ terminal end of the gagpol gene. Linear amplicon was then circularized via Q5 Site Directed Mutagenesis Kit (New England Biolabs) and products were transformed into DH5α. Colonies were obtained and analyzed by restriction enzyme digestion. The gagpo/with deleted integrase (IntDel) is 1224 bp shorter in length than the original gagpol. The correct size plasmid was selected, and the sequence checked by whole plasmid sequencing at Primordium Labs.

FIG. 1 shows that the integrase is part of the gagpol gene which is translated into a polyprotein sequence that gets processed by the viral protease to yield 7 proteins. The integrase (INT) is a 408 aa protein at the C-Terminus of Gag-Pol.

FIG. 2A shows the plasmid map of wild-type GP (GPwt or WT-GP). The Moloney murine leukemia virus (MLV) complete genome is 8332 base pair (bp) (Genbank Accession Number NC_001501). The gagpol is located at nucleotide (nt) 357-5573, the integrase at nt 4347-5573. Note: the gagpol sequence used in this example differs from this reference at nucleotide location 1999, which has a thymine (T) instead of the cytosine (C), resulting in a histidine to tyrosine mutation within the reverse transcriptase. All gagpol constructs used here contain this mutation compared to reference genome NC_001501. This mutation has been reported in a Moloney murine leukemia virus neuropathogenic variant genome, GenBank Acc. Number AF462057.1. FIG. 2B shows a plasmid map of GP-IntDel (or IntDel) in which the integrase coding sequence is deleted.

Table 2 shows titers of vectors with wild-type Gag-Pol, integrase deletion, or double integrase defective mutant, D184AE220A (IDRV2-2) with RVE and GMCSF-vTK payload.

TABLE 2
Titers of vectors with wild-type Gag-Pol, integrase deletion,
or double integrase defective mutant, D184AE220A (IDRV2-2)

| Vectors with GMCSF-vTK and RVE | |
|---|---|
| (amphotropic 4070A envelope) | Vector Genome Copies/mL |
| WT-GP | 6.19E+08 |
| GP-IntDel | 4.28E+08 |
| IDRV2-2 | 6.16E+08 |

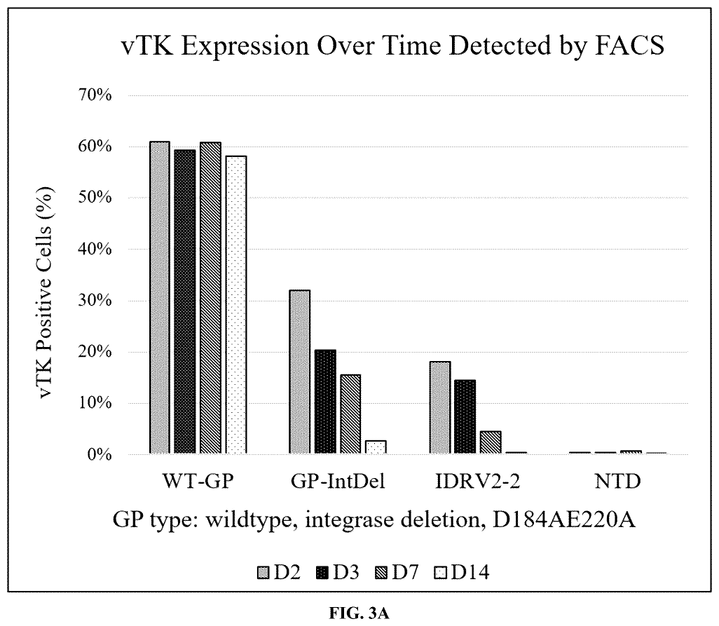

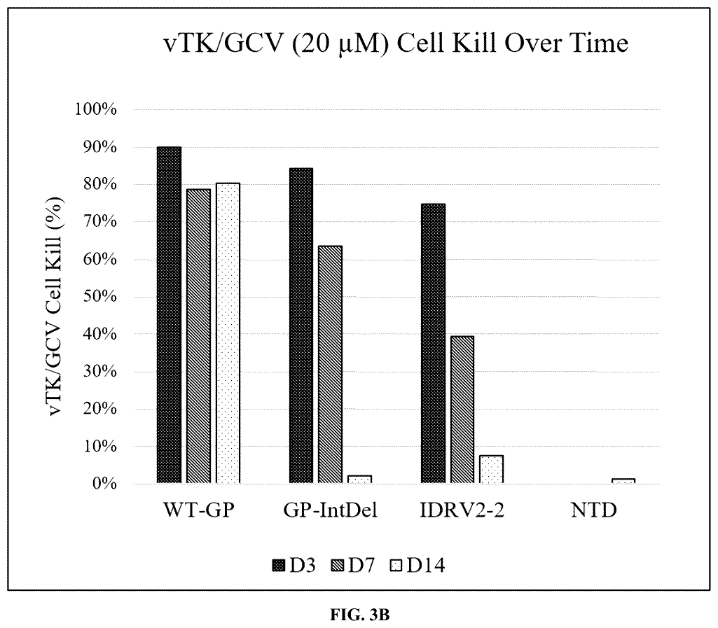

GP-IntDel capacity for generating vector particles was assessed by transient triple transfection of 293T cells with either WT-GP (GP340-VKS), GP-IntDel, or IDRV2-2 (D184AE220A) along with RVE (RetroVector Envelope, amphotropic 4070A) and GMCSF-HSV1-TK payload. Vector supernatants were titered by RT-qPCR. As shown in Table 2, the resulting GP-IntDel vector titers were comparable to those generated using WT-GP and IDRV2-2, suggesting the deletion of the integrase did not affect ability to assemble vector particles. FIG. 3A vTK expression detected by FACS on Day 2, Day 3, Day 7, and Day 14 post-transduction of A375 cells with vectors with WT-GP, GP-IntDel, or IDRV2-2. FIG. 3B shows vTK/GCV cell kill activity on Day 3, Day 7, and Day 14 post-transduction with vectors with WT-GP, GP-IntDel, or IDRV2-2.

A375 human melanoma cells were transduced with WT-GP, GP-IntDel, or IDRV2-2GMCSF-vTK vector supernatants at le8 vector genome copies/mL in 1 mL total media for 4 hours, followed by addition of 1 mL growth medium. FACS was carried out by staining for vTK with a FITC-conjugated antibody at days 2, 3, 7, and 14 post-transduction. Consistent expression of vTK was seen in cells transduced with WT vector, as expected. IntDel and IDRV2-2 showed an initial burst of expression at early time points followed by a steady drop down to non-transduced background levels by Day 14. Cell kill experiments were also carried out at days 3, 7 and 14 post-transduction. The cells were harvested and seeded in 96-well plates, 1e4 cells/well/100 μL medium, 6 wells per cell transduction type. Then 3 wells each received 100 μL of 40 μM GCV prepared in growth medium (to make a final concentration of 20 μM), and 3 remaining wells received 100 μL of growth medium. Cells were incubated at 37° C./5% CO₂ with 80% humidity. After 3 days of GCV treatment, the number of remaining live cells was determined by incubating the cells 2 hours with the viability marker PrestoBlue, and relative fluorescence was measured with SpectraMax M5 plate reader (Molecular Devices). For WT-GP, the initial cell kill level remained consistent over the course of 14 days while cells transduced with GP-IntDel vector showed high initial cell kill followed by a decline, similar to that seen with cells transduced with IDRV2-2 vector. This suggested that GP-IntDel system is able to achieve significant expression after initial transduction and that expression is gradually lost over time as the payload is not integrated into the host genome, as is seen in IDRV system. The levels of expression of vTK and cell kill are comparable to those seen in IDRV system.

Table 3 shows titers of vectors generated with wild-type Gag-Pol, integrase deletion, integrase defective IDRV1 (D184A), IDRV2 (D125AD184A), or IDRV2-2 (D184AE220A), along with RVE and copGFP payload.

TABLE 3
Titers of vectors generated with wild-type Gag-Pol,
integrase deletion, integrase defective IDRV1 (D184A),
IDRV2 (D125AD184A), or IDRV2-2 (D184AE220A)

| Vectors with copGFP and RVE | Vector Genome copies/mL |
|---|---|
| WT-GP | 1.73E+08 |
| GP-IntDel | 2.15E+08 |
| IDRV1 | 4.34E+08 |
| IDRV2 | 1.79E+08 |
| IDRV2-2 | 7.29E+08 |

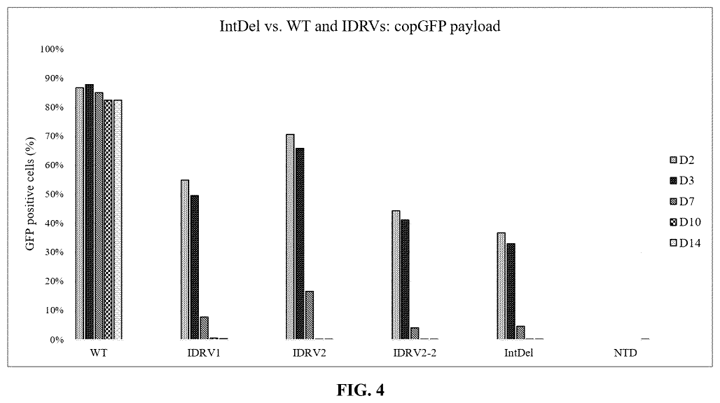

GP-IntDel capacity for generating vector particles was assessed by transient triple transfection of 293T cells with either WT-GP (GP340-VKS), GP-IntDel, IDRV1 (D184A), IDRV2 (D125AD184A), or IDRV2-2 (D184AE220A) along with RVE and copGFP payload. Vector supernatants were titered by RT-qPCR. As shown in Table 3, the resulting GP-IntDel vector titer was comparable to those generated using WT-GP, and other IDRV mutants, indicating deletion or mutation of the integrase does not significantly affect vector particle formation. A375 cells were transduced with 7.5e7 vector genome copies/mL of each vector. Population percentage of copGFP positive cells was assessed by FACS. WT-GP copGFP expression remained constant throughout the assay duration (Day 14 post-transduction). Some variation in the number of copGFP positive cells was seen between all IDRVs. GP-IntDel copGFP expression was comparable to IDRV2-2. The same pattern of expression was seen in GP-IntDel compared to IDRVs with an initial high amount of expression followed by gradual decline and disappearance of expression by Day 14 post-transduction. This data is shown in FIG. 4.

### Example 2: Cell Kill Activity of Viral Vectors Generated from Integrase Deletion in Several Human Cell Lines

Expression and activity of payload, e.g., HSV1-TK of viral vectors generated from GPwt, integrase-defective IDRV2, and integrase-deleted IntDel, was investigated in several cell lines in this example.

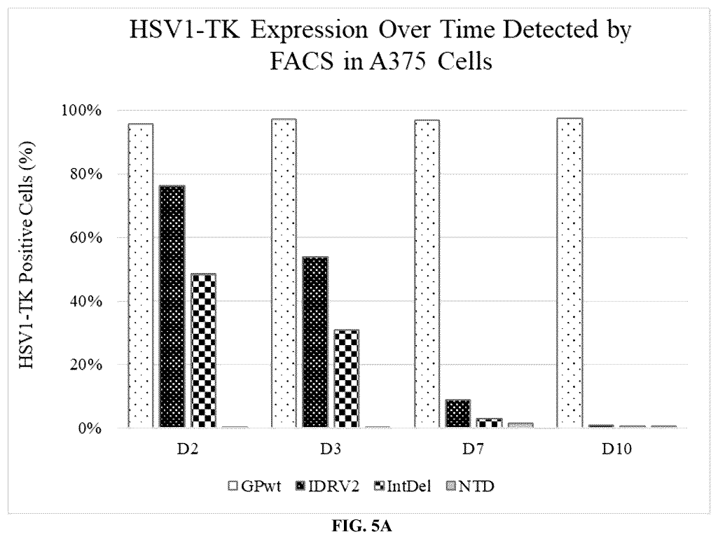

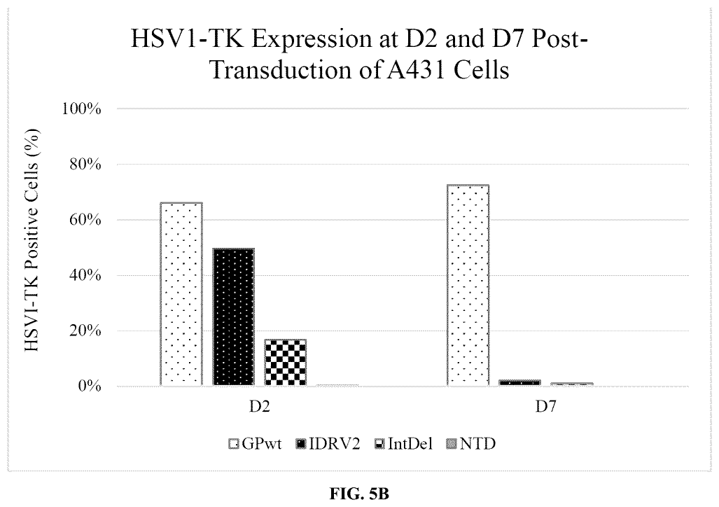

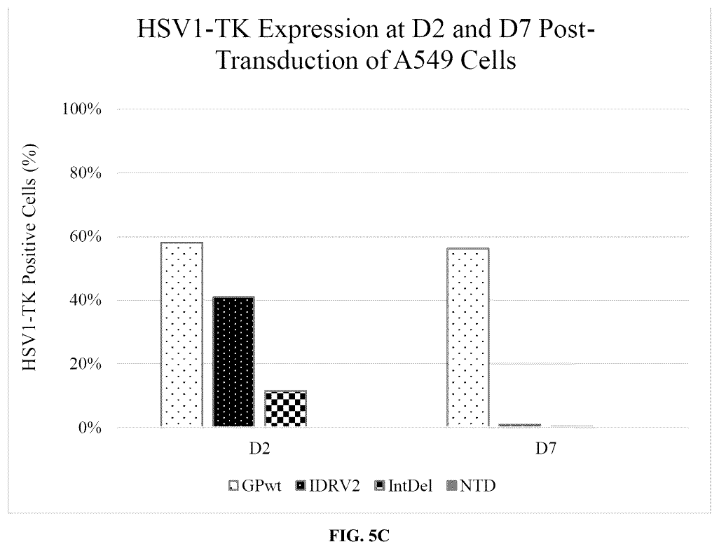

Expression from Int-Del with a HSV1-TK payload was assessed in various cell lines by FACS and experiments were performed as described in the previous example. FIGS. 5A-5C show payload HSV1-TK protein expression detected by flow cytometry over time post-transduction of A375 melanoma cells (FIG. 5A), A431 epidermoid carcinoma calls (FIG. 5B) and A459 lung carcinoma cells (FIG. 5C) transduced with vectors generated with GPwt, integrase-defective IDRV2 and integrase-deleted gagpol (IntDel). IntDel behaves similarly to integrase-defective IDRV2 although expression is reduced compared to integrase-defective IDRV2. NTDs are control non-transduced cells.

Each cell line was transduced with retroviral vectors generated with GPwt, integrase-deleted gagpol (IntDel), and integrase-defective IDRV2, as described in the previous examples. At given time points, cells were collected and seeded to test payload activity (HSV1-TK) by adding GCV and determining cell kill % after 3 days of treatment with GCV. NTDs are non-transduced cells.

Cell kill experiments were carried out as described in the previous example, at days 3, 7 and 14 post-transduction in A375 melanoma cells, A431 epidermoid carcinoma cells, A549 lung carcinoma cells, and U87MG malignant brain glioma cells. Day 17 was also accessed in U87MG cells.

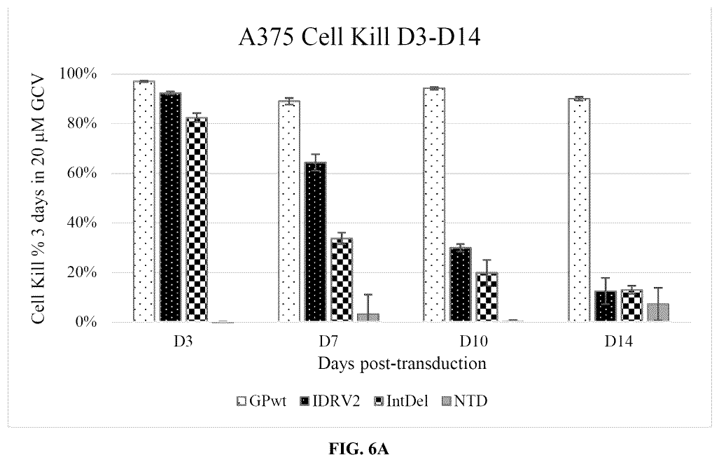

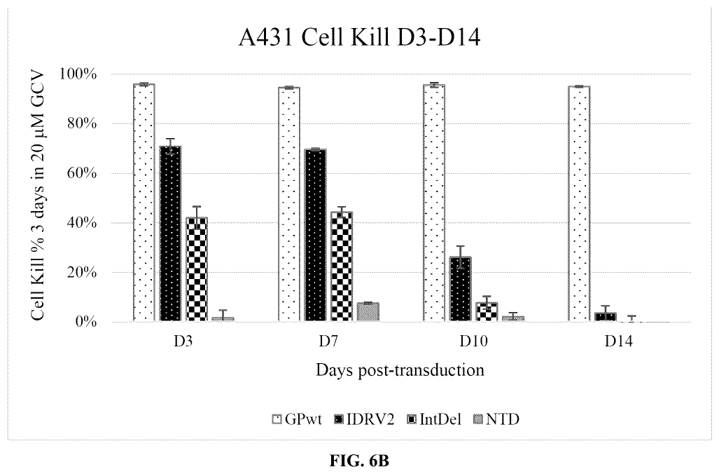

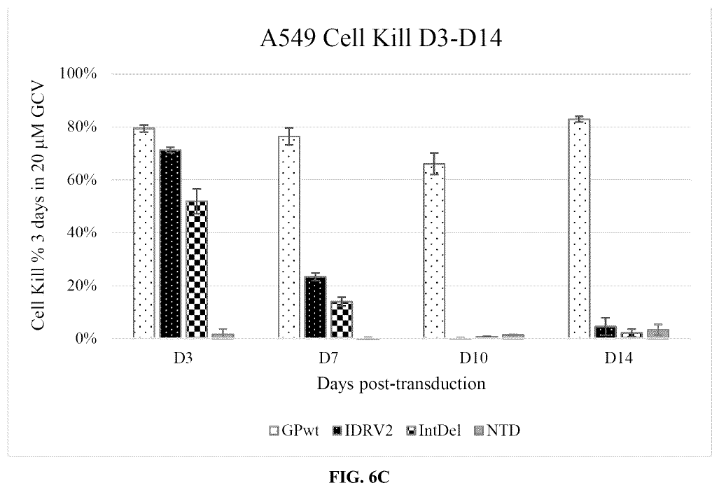

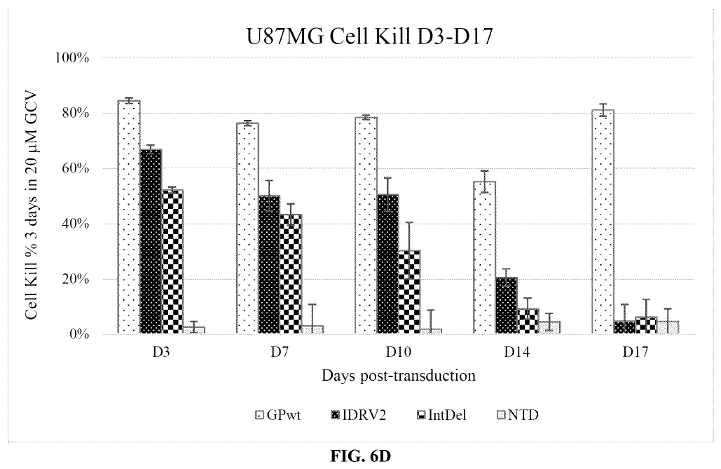

FIGS. 6A-6D show HSV1-TK/GCV cell kill activity over time post-transduction of A375 (FIG. 6A), A431 (FIG. 6B), A549 (FIG. 6C) and U87MG (FIG. 6D) with vectors generated with GPwt, or IntDel, or IDRV2.

Cell kill percentage decreased over time similarly in cells transduced with integrase-deleted or integrase-defective retroviral vectors, while remaining high for those cells that were transduced with the integrating GPwt retroviral vectors.

### Example 3. SARS-CoV2 Omicron Payload

In this example, A375 cells were transduced with retroviral vectors generated with GPwt, IDRV2, or IntDel as described in the previous examples with a SARS-CoV2 Omicron payload.

A375 cells were transduced for 4 hours in the presence of 8 μg/mL polybrene. Conditioned media samples from each transduction, containing the secreted payload protein, were collected at Day 2, 3, 7 and 14 post-transduction, and analyzed by Western blotting with anti-SARS-CoV2 Spike antibody. NTDs are control non-transduced A375 cells.

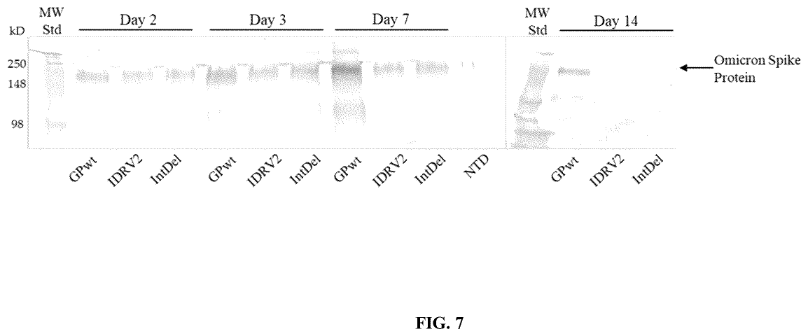

FIG. 7, shows a Western blot detection of expression of SARS-CoV2 Omicron Spike protein secreted over time by A375 cells transduced with vectors made with GPwt, IDRV2, and IntDel, respectively. For cells transduced with IntDel or IDRV2 retroviral vectors, payload expression increased between D2 and D3, was detectable at D7 but gone by D14, whereas omicron spike remained expressed in cells transduced with the integrating GPwt vector.

### Example 4. Assessment of Integration of Payload

In this example, integration of payload of viral vectors generated from the previous examples was accessed.

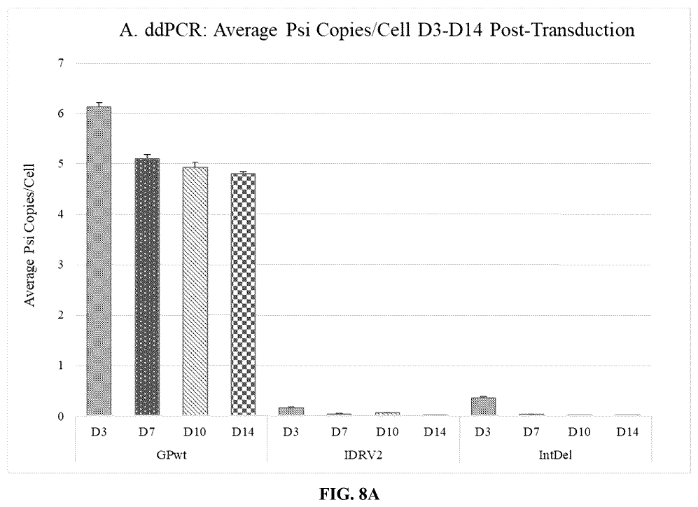

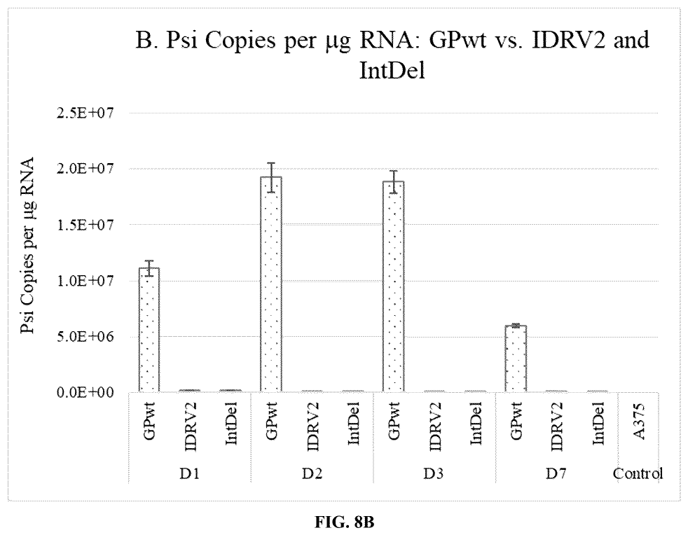

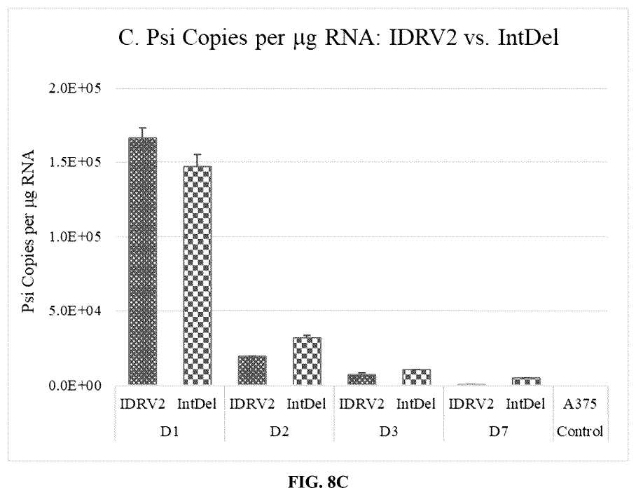

Briefly, whole cell DNA was extracted using QIAcube at Day 3, Day 7, Day 10, and Day 14post-transduction of A375 cells. Copy numbers of Psi signal of integrated payload gene were analyzed by ddPCR. As shown in FIG. 8A, no integrated payload gene could be detected when cells were transduced with the integrase-deleted IntDel or integrase-defective IDRV2 vectors. The limit of detection (LOD) of the assay is 0.031 Psi copy/cell. The low signal seen at Day 3 for viral vectors generated with IDRV2 and IntDel represents episomal and/or circular vector DNA that are formed at early stages post-transduction (Gianni et al. (1975); Maetzig et al. (2011); Steinrigl et al. (2007)) but disappeared over time.

To confirm this result, RNA was also extracted at Day 1, Day 2, Day 3, and Day 7 post-transduction of A375 cells, using QIAcube. Copy numbers of Psi signal of payload RNA were analyzed by RT-qPCR. FIGS. 8B-8C show payload RNA over time, accessed by RT-qPCR in cells transduced with vectors generated with GPwt versus IDRV2 and IntDel. FIG. 8C is an expansion of figure FIG. 8B without the GPwt to show that there are similar levels of Psi copies/μg RNA in viral vectors generated with IDRV2 and IntDel and that they disappear over time.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

SEQUENCES

>Amino acid sequence of wild type MLV gagpol (GPwt)(1738 aa):
MGQTVTTPLSLTLGHWKDVERIAHNQSVDVKKRRWVTFCSAEWPTFNVGWPRDGTFNRD
LITQVKIKVFSPGPHGHPDQVPYIVTWEALAFDPPPWVKPFVHPKPPPPLPPSAPSLPLEPPRS
TPPRSSLYPALTPSLGAKPKPQVLSDSGGPLIDLLTEDPPPYRDPRPPPSDRDGNGGEATPAG
EAPDPSPMASRLRGRREPPVADSTTSQAFPLRAGGNGQLQYWPFSSSDLYNWKNNNPSFSE
DPGKLTALIESVLITHQPTWDDCQQLLGTLLTGEEKQRVLLEARKAVRGDDGRPTQLPNEV
DAAFPLERPDWDYTTQAGRNHLVHYRQLLLAGLQNAGRSPTNLAKVKGITQGPNESPSAFL
ERLKEAYRRYTPYDPEDPGQETNVSMSFIWQSAPDIGRKLERLEDLKNKTLGDLVREAEKIF
NKRETPEEREERIRRETEEKEERRRTEDEQKEKERDRRRHREMSKLLATVVSGQKQDRQGG
ERRRSQLDRDQCAYCKEKGHWAKDCPKKPRGPRGPRPQTSLLTLDD*GGQGQEPPPEPRIT
LKVGGQPVTFLVDTGAQHSVLTQNPGPLSDKSAWVQGATGGKRYRWTTDRKVHLATGKV
THSFLHVPDCPYPLLGRDLLTKLKAQIHFEGSGAQVMGPMGQPLQVLTLNIEDEYRLHETS
KEPDVSLGSTWLSDFPQAWAETGGMGLAVRQAPLIIPLKATSTPVSIKQYPMSQEARLGIKP
HIQRLLDQGILVPCQSPWNTPLLPVKKPGTNDYRPVQDLREVNKRVEDIHPTVPNPYNLLSG
LPPSHQWYTVLDLKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNLGYRASAKKAQI
CQKQVKYLGYLLKEGQRWLTEARKETVMGQPTPKTPRQLREFLGTAGFCRLWIPGFAEMA
APLYPLTKTGTLFNWGPDQQKAYQEIKQALLTAPALGLPDLTKPFELFVDEKQGYAKGVLT
QKLGPWRRPVAYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEGLQHNCLDILAEA
HGTRPDLTDQPLPDADHTWYTDGSSLLQEGQRKAGAAVTTETEVIWAKALPAGTSAQRAE
LIALTQALKMAEGKKLNVYTDSRYAFATAHIHGEIYRRRGLLTSEGKEIKNKDEILALLKAL
FLPKRLSIIHCPGHQKGHSAEARGNRMADQAARKAAITETPDTSTLLIENSSPYTSEHFHYTV
TDIKDLTKLGAIYDKTKKYWVYQGKPVMPDQFTFELLDFLHQLTHLSFSKMKALLERSHSP
YYMLNRDRTLKNITETCKACAQVNASKSAVKQGTRVRGHRPGTHWEIDFTEIKPGLYGYK
YLLVFIDTFSGWIEAFPTKKETAKVVTKKLLEEIFPRFGMPQVLGTDNGPAFVSKVSQTVAD
LLGIDWKLHCAYRPQSSGQVERMNRTIKETLTKLTLATGSRDWVLLLPLALYRARNTPGPH
GLTPYEILYGAPPPLVNFPDPDMTRVTNSPSLQAHLQALYLVQHEVWRPLAAAYQEQLDRP
VVPHPYRVGDTVWVRRHQTKNLEPRWKGPYTVLLTTPTALKVDGIAAWIHAAHVKAADP
GGGPSSRLTWRVQRSQNPLKIRLTREAP (SEQ ID NO: 1)
>DNA sequence of wild type MLV gagpol (GPwt):
atgggccagactgttaccactcccttaagtttgaccttaggtcactggaaagatgtcgagcggatcgctcacaaccagtcggtagatgtcaagaag
agacgttgggttaccttctgctctgcagaatggccaacctttaacgtcggatggccgcgagacggcacctttaaccgagacctcatcacccaggtt
aagatcaaggtcttttcacctggcccgcatggacacccagaccaggtcccctacatcgtgacctgggaagccttggcttttgacccccctccctgg
gtcaagccctttgtacaccctaagcctccgcctcctcttcctccatccgccccgtctctcccccttgaacctcctcgttcgaccccgcctcgatcctcc
ctttatccagccctcactccttctctaggcgccaaacctaaacctcaagttctttctgacagtggggggccgctcatcgacctacttacagaagaccc
cccgccttatagggacccaagaccacccccttccgacagggacggaaatggtggagaagcgacccctgcgggagaggcaccggacccctcc
ccaatggcatctcgcctacgtgggagacgggagccccctgtggccgactccactacctcgcaggcattccccctccgcgcaggaggaaacgg
acagcttcaatactggccgttctcctcttctgacctttacaactggaaaaataataacccttctttttctgaagatccaggtaaactgacagctctgatc
gagtctgttctcatcacccatcagcccacctgggacgactgtcagcagctgttggggactctgctgaccggagaagaaaaacaacgggtgctctt
agaggctagaaaggcggtgcggggcgatgatgggcgccccactcaactgcccaatgaagtcgatgccgcttttcccctcgagcgcccagactg
ggattacaccacccaggcaggtaggaaccacctagtccactatcgccagttgctcctagcgggtctccaaaacgcgggcagaagccccaccaa
tttggccaaggtaaaaggaataacacaagggcccaatgagtctccctcggccttcctagagagacttaaggaagcctatcgcaggtacactcctt
atgaccctgaggacccagggcaagaaactaatgtgtctatgtctttcatttggcagtctgccccagacattgggagaaagttagagaggttagaag
atttaaaaaacaagacgcttggagatttggttagagaggcagaaaagatctttaataaacgagaaaccccggaagaaagagaggaacgtatcag
gagagaaacagaggaaaaagaagaacgccgtaggacagaggatgagcagaaagagaaagaaagagatcgtaggagacatagagagatga
gcaagctattggccactgtcgttagtggacagaaacaggatagacagggaggagaacgaaggaggtcccaactcgatcgcgaccagtgtgcct
actgcaaagaaaaggggcactgggctaaagattgtcccaagaaaccacgaggacctcggggaccaagaccccagacctccctcctgacccta
gatgactagggaggtcagggtcaggagcccccccctgaacccaggataaccctcaaagtcggggggcaacccgtcaccttcctggtagatact
ggggcccaacactccgtgctgacccaaaatcctggacccctaagtgataagtctgcctgggtccaaggggctactggaggaaagcggtatcgct
ggaccacggatcgcaaagtacatctagctaccggtaaggtcacccactctttcctccatgtaccagactgtccctatcctctgttaggaagagatttg
ctgactaaactaaaagcccaaatccactttgagggatcaggagctcaggttatgggaccaatggggcagcccctgcaagtgttgaccctaaatat
agaagatgagtatcggctacatgagacctcaaaagagccagatgtttctctagggtccacatggctgtctgattttcctcaggcctgggcggaaac
cgggggcatgggactggcagttcgccaagctcctctgatcatacctctgaaagcaacctctacccccgtgtccataaaacaataccccatgtcac
aagaagccagactggggatcaagccccacatacagagactgttggaccagggaatactggtaccctgccagtccccctggaacacgcccctgc
tacccgttaagaaaccagggactaatgattataggcctgtccaggatctgagagaagtcaacaagcgggtggaagacatccaccccaccgtgcc
caacccttacaacctcttgaggggctcccaccgtcccaccagtggtacactgtgcttgatttaaaggatgcctttttctgcctgagactccacccca
ccagtcagcctctcttcgcctttgagtggagagatccagagatgggaatctcaggacaattgacctggaccagactcccacagggtttcaaaaac
agtcccaccctgtttgatgaggcactgcacagagacctagcagacttccggatccagcacccagacttgatcctgctacagtacgtggatgactta
ctgctggccgccacttctgagctagactgccaacaaggtactcgggccctgttacaaaccctagggaacctcgggtatcgggcctcggccaaga
aagcccaaatttgccagaaacaggtcaagtatctggggtatcttctaaaagagggtcagagatggctgactgaggccagaaaagagactgtgat
ggggcagcctactccgaagacccctcgacaactaagggagttcctagggacggcaggcttctgtcgcctctggatccctgggtttgcagaaatg
gcagcccccttgtaccctctcaccaaaacggggactctgtttaattggggcccagaccaacaaaaggcctatcaagaaatcaagcaagctcttcta
actgccccagccctggggttgccagatttgactaagccctttgaactctttgtcgacgagaagcagggctacgccaaaggtgtcctaacgcaaaa
actgggaccttggcgtcggccggtggcctacctgtccaaaaagctagacccagtagcagctgggtggcccccttgcctacggatggtagcagc
cattgccgtactgacaaaggatgcaggcaagctaaccatgggacagccactagtcattctggccccccatgcagtagaggcactagtcaaacaa
ccccccgaccgctggctttccaacgcccggatgactcactatcaggccttgcttttggacacggaccgggtccagttcggaccggtggtagccct
gaacccggctacgctgctcccactgcctgaggaagggctgcaacacaactgccttgatatcctggccgaagcccacggaacccgacccgacct
aacggaccagccgctcccagacgccgaccacacctggtacacggatggaagcagtctcttacaagagggacagcgtaaggcgggagctgcg
gtgaccaccgagaccgaggtaatctgggctaaagccctgccagccgggacatccgctcagcgggctgaactgatagcactcacccaggccct
aaagatggcagaaggtaagaagctaaatgtttatactgatagccgttatgcttttgctactgcccatatccatggagaaatatacagaaggcgtggg
ttgctcacatcagaaggcaaagagatcaaaaataaagacgagatcttggccctactaaaagccctctttctgcccaaaagacttagcataatccatt
gtccaggacatcaaaagggacacagcgccgaggctagaggcaaccggatggctgaccaagcggcccgaaaggcagccatcacagagactc
cagacacctctaccctcctcatagaaaattcatcaccctacacctcagaacattttcattacacagtgactgatataaaggacctaaccaagttgggg
gccatttatgataaaacaaagaagtattgggtctaccaaggaaaacctgtgatgcctgaccagtttacttttgaattattagactttcttcatcagctga
ctcacctcagcttctcaaaaatgaaggctctcctagagagaagccacagtccctactacatgctgaaccgggatcgaacactcaaaaatatcactg
agacctgcaaagcttgtgcacaagtcaacgccagcaagtctgccgttaaacagggaactagggtccgcgggcatcggcccggcactcattggg
agatcgatttcaccgagataaagcccggattgtatggctataaatatcttctagtttttatagataccttttctggctggatagaagccttcccaaccaa
gaaagaaaccgccaaggtcgtaaccaagaagctactagaggagatcttccccaggttcggcatgcctcaggtattgggaactgacaatgggcct
gccttcgtctccaaggtgagtcagacagtggccgatctgttggggattgattggaaattacattgtgcatacagaccccaaagctcaggccaggta
gaaagaatgaatagaaccatcaaggagactttaactaaattaacgcttgcaactggctctagagactgggtgctcctactccccttagccctgtacc
gagcccgcaacacgccgggcccccatggcctcaccccatatgagatcttatatggggcacccccgccccttgtaaacttccctgaccctgacatg
acaagagttactaacagcccctctctccaagctcacttacaggctctctacttagtccagcacgaagtctggagacctctggcggcagcctaccaa
gaacaactggaccgaccggtggtacctcacccttaccgagtcggcgacacagtgtgggtccgccgacaccagactaagaacctagaacctcgc
tggaaaggaccttacacagtcctgctgaccacccccaccgccctcaaagtagacggcatcgcagcttggatacacgccgcccacgtgaaggct
gccgaccccgggggtggaccatcctctagactgacatggcgcgttcaacgctctcaaaaccccttaaaaataaggttaacccgcgaggccccct
aa (SEQ ID NO: 2)
>Amino acid sequence of IntDel gagpol (1330 aa):
MGQTVTTPLSLTLGHWKDVERIAHNQSVDVKKRRWVTFCSAEWPTFNVGWPRDGTFNRD
LITQVKIKVFSPGPHGHPDQVPYIVTWEALAFDPPPWVKPFVHPKPPPPLPPSAPSLPLEPPRS
TPPRSSLYPALTPSLGAKPKPQVLSDSGGPLIDLLTEDPPPYRDPRPPPSDRDGNGGEATPAG
EAPDPSPMASRLRGRREPPVADSTTSQAFPLRAGGNGQLQYWPFSSSDLYNWKNNNPSFSE
DPGKLTALIESVLITHQPTWDDCQQLLGTLLTGEEKQRVLLEARKAVRGDDGRPTQLPNEV
DAAFPLERPDWDYTTQAGRNHLVHYRQLLLAGLQNAGRSPTNLAKVKGITQGPNESPSAFL
ERLKEAYRRYTPYDPEDPGQETNVSMSFIWQSAPDIGRKLERLEDLKNKTLGDLVREAEKIF
NKRETPEEREERIRRETEEKEERRRTEDEQKEKERDRRRHREMSKLLATVVSGQKQDRQGG
ERRRSQLDRDQCAYCKEKGHWAKDCPKKPRGPRGPRPQTSLLTLDD*GGQGQEPPPEPRIT
LKVGGQPVTFLVDTGAQHSVLTQNPGPLSDKSAWVQGATGGKRYRWTTDRKVHLATGKV
THSFLHVPDCPYPLLGRDLLTKLKAQIHFEGSGAQVMGPMGQPLQVLTLNIEDEYRLHETS
KEPDVSLGSTWLSDFPQAWAETGGMGLAVRQAPLIIPLKATSTPVSIKQYPMSQEARLGIKP
HIQRLLDQGILVPCQSPWNTPLLPVKKPGTNDYRPVQDLREVNKRVEDIHPTVPNPYNLLSG
LPPSHQWYTVLDLKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNLGYRASAKKAQI
CQKQVKYLGYLLKEGQRWLTEARKETVMGQPTPKTPRQLREFLGTAGFCRLWIPGFAEMA
APLYPLTKTGTLFNWGPDQQKAYQEIKQALLTAPALGLPDLTKPFELFVDEKQGYAKGVLT
QKLGPWRRPVAYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEGLQHNCLDILAEA
HGTRPDLTDQPLPDADHTWYTDGSSLLQEGQRKAGAAVTTETEVIWAKALPAGTSAQRAE
LIALTQALKMAEGKKLNVYTDSRYAFATAHIHGEIYRRRGLLTSEGKEIKNKDEILALLKAL
FLPKRLSIIHCPGHQKGHSAEARGNRMADQAARKAAITETPDTSTLL (SEQ ID NO: 3)
> DNA sequence of IntDel gagpol:
atgggccagactgttaccactcccttaagtttgaccttaggtcactggaaagatgtcgagcggatcgctcacaaccagtcggtagatgtcaagaag
agacgttgggttaccttctgctctgcagaatggccaacctttaacgtcggatggccgcgagacggcacctttaaccgagacctcatcacccaggtt
aagatcaaggtcttttcacctggcccgcatggacacccagaccaggtcccctacatcgtgacctgggaagccttggcttttgacccccctccctgg
gtcaagccctttgtacaccctaagcctccgcctcctcttcctccatccgccccgtctctcccccttgaacctcctcgttcgaccccgcctcgatcctcc
ctttatccagccctcactccttctctaggcgccaaacctaaacctcaagttctttctgacagtggggggccgctcatcgacctacttacagaagaccc
cccgccttatagggacccaagaccacccccttccgacagggacggaaatggtggagaagcgacccctgcgggagaggcaccggacccctcc
ccaatggcatctcgcctacgtgggagacgggagccccctgtggccgactccactacctcgcaggcattccccctccgcgcaggaggaaacgg
acagcttcaatactggccgttctcctcttctgacctttacaactggaaaaataataacccttctttttctgaagatccaggtaaactgacagctctgatc
gagtctgtcctcatcacccatcagcccacctgggacgactgtcagcagctgttggggactctgctgaccggagaagaaaaacaacgggtgctctt
agaggctagaaaggcggtgcggggcgatgatgggcgccccactcaactgcccaatgaagtcgatgccgcttttcccctcgagcgcccagactg
ggattacaccacccaggcaggtaggaaccacctagtccactatcgccagttgctcctagcgggtctccaaaacgcgggcagaagccccaccaa
tttggccaaggtaaaaggaataacacaagggcccaatgagtctccctcggccttcctagagagacttaaggaagcctatcgcaggtacactcctt
atgaccctgaggacccagggcaagaaactaatgtgtctatgtctttcatttggcagtctgccccagacattgggagaaagttagagaggttagaag
atttaaaaaacaagacgcttggagatttggttagagaggcagaaaagatctttaataaacgagaaaccccggaagaaagagaggaacgtatcag
gagagaaacagaggaaaaagaagaacgccgtaggacagaggatgagcagaaagagaaagaaagagatcgtaggagacatagagagatga
gcaagctattggccactgtcgttagtggacagaaacaggatagacagggaggagaacgaaggaggtcccaactcgatcgcgaccagtgtgcct
actgcaaagaaaaggggcactgggctaaagattgtcccaagaaaccacgaggacctggggaccaagaccccagacctccctcctgacccta
gatgactagggaggtcagggtcaggagcccccccctgaacccaggataaccctcaaagtcggggggcaacccgtcaccttcctggtagatact
ggggcccaacactccgtgctgacccaaaatcctggacccctaagtgataagtctgcctgggtccaaggggctactggaggaaagcggtatcgct
ggaccacggatcgcaaagtacatctagctaccggtaaggtcacccactctttcctccatgtaccagactgtccctatcctctgttaggaagagatttg
ctgactaaactaaaagcccaaatccactttgagggatcaggagctcaggttatgggaccaatggggcagcccctgcaagtgttgaccctaaatat
agaagatgagtatcggctacatgagacctcaaaagagccagatgtttctctagggtccacatggctgtctgattttcctcaggcctgggcggaaac
cgggggcatgggactggcagttcgccaagctcctctgatcatacctctgaaagcaacctctacccccgtgtccataaaacaataccccatgtcac
aagaagccagactggggatcaagccccacatacagagactgttggaccagggaatactggtaccctgccagtccccctggaacacgcccctgc
tacccgttaagaaaccagggactaatgattataggcctgtccaggatctgagagaagtcaacaagcgggtggaagacatccaccccaccgtgcc
caacccttacaacctcttgagcgggctcccaccgtcccaccagtggtacactgtgcttgatttaaaggatgcctttttctgcctgagactccacccca
ccagtcagcctctcttcgcctttgagtggagagatccagagatgggaatctcaggacaattgacctggaccagactcccacagggtttcaaaaac
agtcccaccctgtttgatgaggcactgcacagagacctagcagacttccggatccagcacccagacttgatcctgctacagtacgtggatgactta
ctgctggccgccacttctgagctagactgccaacaaggtactcgggccctgttacaaaccctagggaacctcgggtatcgggcctcggccaaga
aagcccaaatttgccagaaacaggtcaagtatctggggtatcttctaaaagagggtcagagatggctgactgaggccagaaaagagactgtgat
ggggcagcctactccgaagacccctcgacaactaagggagttcctagggacggcaggcttctgtcgcctctggatccctgggtttgcagaaatg
gcagcccccttgtaccctctcaccaaaacggggactctgtttaattggggcccagaccaacaaaaggcctatcaagaaatcaagcaagctcttcta
actgccccagccctggggttgccagatttgactaagccctttgaactctttgtcgacgagaagcagggctacgccaaaggtgtcctaacgcaaaa
actgggaccttggcgtcggccggtggcctacctgtccaaaaagctagacccagtagcagctgggtggcccccttgcctacggatggtagcagc
cattgccgtactgacaaaggatgcaggcaagctaaccatgggacagccactagtcattctggccccccatgcagtagaggcactagtcaaacaa
ccccccgaccgctggctttccaacgcccggatgactcactatcaggccttgcttttggacacggaccgggtccagttcggaccggtggtagccct
gaacccggctacgctgctcccactgcctgaggaagggctgcaacacaactgccttgatatcctggccgaagcccacggaacccgacccgacct
aacggaccagccgctcccagacgccgaccacacctggtacacggatggaagcagtctcttacaagagggacagcgtaaggcgggagctgcg
gtgaccaccgagaccgaggtaatctgggctaaagccctgccagccgggacatccgctcagcgggctgaactgatagcactcacccaggccct
aaagatggcagaaggtaagaagctaaatgtttatactgatagccgttatgcttttgctactgcccatatccatggagaaatatacagaaggcgtggg
ttgctcacatcagaaggcaaagagatcaaaaataaagacgagatcttggccctactaaaagccctctttctgcccaaaagacttagcataatccatt
gtccaggacatcaaaagggacacagcgccgaggctagaggcaaccggatggctgaccaagcggcccgaaaggcagccatcacagagactc
cagacacctctaccctcctc (SEQ ID NO: 4)
>Amino acid sequence of wild type integrase:
IENSSPYTSEHFHYTVTDIKDLTKLGAIYDKTKKYWVYQGKPVMPDQFTFELLDFLHQLTHL
SFSKMKALLERSHSPYYMLNRDRTLKNITETCKACAQVNASKSAVKQGTRVRGHRPGTHW
EIDFTEIKPGLYGYKYLLVFIDTFSGWIEAFPTKKETAKVVTKKLLEEIFPRFGMPQVLGTDN
GPAFVSKVSQTVADLLGIDWKLHCAYRPQSSGQVERMNRTIKETLTKLTLATGSRDWVLLL
PLALYRARNTPGPHGLTPYEILYGAPPPLVNFPDPDMTRVTNSPSLQAHLQALYLVQHEVW
RPLAAAYQEQLDRPVVPHPYRVGDTVWVRRHQTKNLEPRWKGPYTVLLTTPTALKVDGIA
AWIHAAHVKAADPGGGPSSRLTWRVQRSQNPLKIRLTREAP (SEQ ID NO: 5)
>DNA sequence of wild type integrase:
atagaaaattcatcaccctacacctcagaacattttcattacacagtgactgatataaaggacctaaccaagttgggggccatttatgataaaacaaa
gaagtattgggtctaccaaggaaaacctgtgatgcctgaccagtttacttttgaattattagactttcttcatcagctgactcacctcagcttctcaaaaa
tgaaggctctcctagagagaagccacagtccctactacatgctgaaccgggatcgaacactcaaaaatatcactgagacctgcaaagcttgtgca
caagtcaacgccagcaagtctgccgttaaacagggaactagggtccgcgggcatcggcccggcactcattgggagatcgatttcaccgagata
aagcccggattgtatggctataaatatcttctagtttttatagataccttttctggctggatagaagccttcccaaccaagaaagaaaccgccaaggtc
gtaaccaagaagctactagaggagatcttccccaggttcggcatgcctcaggtattgggaactgacaatgggcctgccttcgtctccaaggtgagt
cagacagtggccgatctgttggggattgattggaaattacattgtgcatacagaccccaaagctcaggccaggtagaaagaatgaatagaaccat
caaggagactttaactaaattaacgcttgcaactggctctagagactgggtgctcctactccccttagccctgtaccgagcccgcaacacgccgg
gcccccatggcctcaccccatatgagatcttatatggggcacccccgccccttgtaaacttccctgaccctgacatgacaagagttactaacagcc
cctctctccaagctcacttacaggctctctacttagtccagcacgaagtctggagacctctggcggcagcctaccaagaacaactggaccgaccg
gtggtacctcacccttaccgagtcggcgacacagtgtgggtccgccgacaccagactaagaacctagaacctcgctggaaaggaccttacaca
gtcctgctgaccacccccaccgccctcaaagtagacggcatcgcagcttggatacacgccgcccacgtgaaggctgccgaccccgggggtgg
accatcctctagactgacatggcgcgttcaacgctctcaaaaccccttaaaaataaggttaacccgcgaggccccc (SEQ ID NO: 6)
>Amino acid sequence of wild type reverse transcriptase:
TLNIEDEYRLHETSKEPDVSLGSTWLSDFPQAWAETGGMGLAVRQAPLIIPLKATSTPVSIKQ
YPMSQEARLGIKPHIQRLLDQGILVPCQSPWNTPLLPVKKPGTNDYRPVQDLREVNKRVEDI
HPTVPNPYNLLSGLPPSHQWYTVLDLKDAFFCLRLHPTSQPLFAFEWRDPEMGISGQLTWT
RLPQGFKNSPTLFDEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREFLGTAG
FCRLWIPGFAEMAAPLYPLTKTGTLFNWGPDQQKAYQEIKQALLTAPALGLPDLTKPFELFV
DEKQGYAKGVLTQKLGPWRRPVAYLSKKLDPVAAGWPPCLRMVAAIAVLTKDAGKLTM
GQPLVILAPHAVEALVKQPPDRWLSNARMTHYQALLLDTDRVQFGPVVALNPATLLPLPEE
GLQHNCLDILAEAHGTRPDLTDQPLPDADHTWYTDGSSLLQEGQRKAGAAVTTETEVIWA
KALPAGTSAQRAELIALTQALKMAEGKKLNVYTDSRYAFATAHIHGEIYRRRGLLTSEGKEI
KNKDEILALLKALFLPKRLSIIHCPGHQKGHSAEARGNRMADQAARKAAITETPDTSTLL
(SEQ ID NO: 7)
>Wild type HSV1-TK:
MASYPGHQHASAFDQAARSRGHSNRRTALRPRRQQEATEVRPEQKMPTLLRVYIDGPHGM
GKTTTTQLLVALGSRDDIVYVPEPMTYWRVLGASETIANIYTTQHRLDQGEISAGDAAVVM
TSAQITMGMPYAVTDAVLAPHIGGEAGSSHAPPPALTLIFDRHPIAALLCYPAARYLMGSMT
PQAVLAFVALIPPTLPGTNIVLGALPEDRHIDRLAKRQRPGERLDLAMLAAIRRVYGLLANT
VRYLQCGGSWREDWGQLSGTAVPPQGAEPQSNAGPRPHIGDTLFTLFRAPELLAPNGDLYN
VFAWALDVLAKRLR (SEQ ID NO: 11)
>Nuclear export sequence:
LQKKLEELELDG (SEQ ID NO: 12)
>Signal peptide:
MDAMKRGLCCVLLLCGAVFVSASQEIHARFRR (SEQ ID NO: 13)
>Extracellular domain of M2 protein (M2e) of influenza A:
MSLLTEVETPIRNEWGCRCNDSSD (SEQ ID NO: 14)
>Full-length Spike protein:
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTLKS
FTVEKGIYQTSNFRVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGENCYFPL
QSYGFQPTNGVGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICASYQTQ
TNSPRRARSVASQSIIAYTMSLGAENSVAYSNNSIAIPTNFTISVTTEILPVSMTKTSVDCTMY
ICGDSTECSNLLLQYGSFCTQLNRALTGIAVEQDKNTQEVFAQVKQIYKTPPIKDFGGFNFSQ
ILPDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRENGIGVTQNVLYENQKLIANQFNS
AIGKIQDSLSSTASALGKLQDVVNQNAQALNTLVKQLSSNFGAISSVLNDILSRLDKVEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISGINASVV
NIQKEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT (SEQ ID NO: 21)
>Omicron mutant Spike protein:
MFVFLVLLPLVSSQCVNLITRTQSYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVI
KVCEFQFCNDPFLDVYYHKNNKSWMESEFRVYSSANNCTFEYVSQPFLMDLEGKQGNFKN
LREFVFKNIDGYFKIYSKHTPINLGRDLPQGFSALEPLVDLPIGINITRFQTLLALHRSYLTPGD
SSSGWTAGAAAYYVGYLQPRTFLLKYNENGTITDAVDCALDPLSETKCTLKSFTVEKGIYQ
TSNFR VQPTESIVRFPNITNLCPFDEVFNATRFASVYAWNRKRISNCVADYSVLYNFAPFFAF
KCYGVSPTKLNDLCFTNVYADSFVIRGNEVSQIAPGQTGNIADYNYKLPDDFTGCVIAWNS
NKLDSKVGGNYNYLYRLFRKSNLKPFERDISTEIYQAGNKPCNGVAGENCYFPLRSYGFRPT
YGVGHQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVA
IHADQLTPTWRVYSTGSNVFQTRAGCLIGAEYVNNSYECDIPIGAGICASYQTQTKSHRRAR
SVASQSIIAYTMSLGAENSVAYSNNSIAIPTNFTISVTTEILPVSMTKTSVDCTMYICGDSTEC
SNLLLQYGSFCTQLKRALTGIAVEQDKNTQEVFAQVKQIYKTPPIKYFGGFNFSQILPDPSKP
SKRSFIEDLLFNKVTLADAGFIKQYGDCLGDIAARDLICAQKFNGLTVLPPLLTDEMIAQYTS
ALLAGTITSGWTFGAGAALQIPFAMQMAYRENGIGVTQNVLYENQKLIANQFNSAIGKIQDS
LSSTASALGKLQDVVNHNAQALNTLVKQLSSKFGAISSVLNDILSRLDKVEAEVQIDRLITG
RLQSLQTYVTQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLCCMTSCCSCLKGC
CSCGSCCKFDEDDSEPVLKGVKLHYT (SEQ ID NO: 22)
>Forward primer:
TAATGAGCGGCCGCGAAG (SEQ ID NO: 24)
>Reverse primer:
GAGGAGGGTAGAGGTGTC (SEQ ID NO: 25)
>Wild-type Sindbis virus E2 Protein
SVIDDFTLTSPYLGTCSYCHHTVPCFSPVKIEQVWDEADDNTIRIQTSAQFGYDQSGAASAN
KYRYMSLKQDHTVKEGTMDDIKISTSGPCRRLSYKGYFLLAKCPPGDSVTVSIVSSNSATSC
TLARKIKPKFVGREKYDLPPVHGKKIPCTVYDRLKETTAGYITMHRPRPHAYTSYLEESSGK
VYAKPPSGKNITYECKCGDYKTGTVSTRTEITGCTAIKQCVAYKSDQTKWVFNSPDLIRHD
DHTAQGKLHLPFKLIPSTCMVPVAHAPNVIHGFKHISLQLDTDHLTLLTTRRLGANPEPTTE
WIVGKTVRNFTVDRDGLEYIWGNHEPVRVYAQESAPGDPHGWPHEIVQHYYHRHPVYTIL
AVASATVAMMIGVTVAVLCACKARRECLTPYALAPNAVIPTSLALLCCVRSANA (SEQ ID
NO: 26)

1-80. (canceled)

81. A method of treating or preventing a disease or condition in a subject, comprising administering to the subject a recombinant retroviral vector comprising a first nucleic acid sequence having a deleted integrase and a second nucleic acid sequence encoding at least one payload, wherein the recombinant retroviral vector has defective retroviral integration activity as compared to a recombinant retroviral vector comprising a wild-type integrase, wherein the at least one payload comprising the antigen induces an immune response in the subject, thereby treating or preventing the disease or condition in the subject.

82. The method of claim 81, wherein the immune response comprises induction of a neutralizing antibody targeting the antigen, thereby generating immunity against the antigen in the subject.

83. The method of claim 81, wherein the immune response comprises induction of an immunoglobulin antibody targeting the antigen, thereby generating immunity against the antigen in the subject.

84. The method of claim 83, wherein the immunoglobulin antibody comprises an IgG antibody, an IgM antibody, an IgA antibody, an IgD antibody, an IgE antibody, or a combination thereof.

85. The method of claim 83, wherein the immunoglobulin antibody comprises an IgG antibody.

86. The method of claim 81, wherein the at least one payload is expressed in the subject for at least one day, at least three days, at least five days, or at least nine days.

87. The method of claim 81, wherein the at least one payload is secreted in the subject for at least one day, at least three days, at least five days, or at least nine days.

88. The method of claim 81, wherein a duration of the immune response induced by the at least one payload is expressed for at least one day, at least three days, at least five days, or at least nine days is increased by at least 10%, at least 20%, at least 50%, at least 100%, at least 5-fold, at least 10-fold, or more compared to a duration of an immune response induced by a comparable payload that is expressed for fewer than one day, three days, five days, or nine days.

89. The method of claim 81, wherein a duration of the immune response induced by the at least one payload is secreted for at least one day, at least three days, at least five days, or at least nine days is increased by at least 10%, at least 20%, at least 50%, at least 100%, at least 5-fold, at least 10-fold, or more compared to a duration of an immune response induced by a comparable payload that is expressed for fewer than one day, three days, five days, or nine days.

90. The method of claim 81, wherein the immune response persists in the subject for at least three months, at least four months, at least five months, at least six months, at least 12 months, or longer.

91. The method of claim 81, wherein the payload is selected from the group consisting of a therapeutic and an antigen.

92. The method of claim 81, wherein the deleted integrase is a deletion of about 12%, about 15%, about 20%, about 30%, about 40%, about 50%, about 60%, about 70%, about 80%, about 90%, about 95%, about 99%, or about 100% of an integrase coding sequence.

93. The method of claim 81, wherein the deleted integrase is a deletion of at least about 80, about 160, about 240, about 320, or about 408 residues of amino acids 1331-1738 of a murine leukemia virus Gag-Pol polyprotein.

94. The method of claim 81, wherein the antigen comprises a pathogen polypeptide or fragment thereof or a cancer polypeptide or fragment thereof.

95. The method of claim 81, wherein the recombinant retroviral vector comprises at least one alphavirus envelope protein.

96. The method of claim 95, wherein the at least one alphavirus envelope protein comprises at least one Sindbis virus envelope protein comprising E3 protein, E2 protein, 6K protein, E1 protein, or a combination thereof.

97. The method of claim 96, wherein the at least one Sindbis virus envelope protein comprises at least one mutation.

98. The method of claim 97, wherein the at least one mutation increases binding affinity between the at least one Sindbis virus envelope protein and a human dendritic cell.

99. The method of claim 98, wherein the at least one mutation is E160G of the E2 protein of the Sindbis virus envelope protein when compared to a wild-type sequence.

100. The method of claim 81, wherein the recombinant retroviral vector comprises at least one modified untranslated region (UTR).


SEQUENCE LISTING (26 sequences)

SEQ ID NO: 1 - protein, 1738 aa, Moloney murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDQGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 2 - DNA, 5217 bp, Moloney murine leukemia virus
  source 1..5217: mol_type = genomic DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaa

SEQ ID NO: 3 - protein, 1330 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDQGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 4 - DNA, 3990 bp, synthetic construct
  source 1..3990: mol_type = other DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgtcctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctc

SEQ ID NO: 5 - protein, 408 aa, unidentified
  source 1..408: note = Integrase
IENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 6 - DNA, 1224 bp, unidentified
  source 1..1224: mol_type = unassigned DNA; note = Integrase
atagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccc

SEQ ID NO: 7 - protein, 671 aa, unidentified
  source 1..671: note = Reverse transcriptase
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 8-10 - skipped (3 entries)

SEQ ID NO: 11 - protein, 320 aa, Human alphaherpesvirus 1
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLR

SEQ ID NO: 12 - protein, 12 aa, synthetic construct
LQKKLEELELDG

SEQ ID NO: 13 - protein, 32 aa, synthetic construct
MDAMKRGLCCVLLLCGAVFVSASQEIHARFRR

SEQ ID NO: 14 - protein, 24 aa, Influenza A virus
MSLLTEVETPIRNEWGCRCNDSSD

SEQ ID NO: 15-20 - skipped (6 entries)

SEQ ID NO: 21 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1270 aa, Severe acute respiratory syndrome coronavirus 2
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - skipped

SEQ ID NO: 24 - DNA, 18 bp, synthetic construct
  source 1..18: mol_type = other DNA
taatgagcggccgcgaag

SEQ ID NO: 25 - DNA, 18 bp, synthetic construct
  source 1..18: mol_type = other DNA
gaggagggtagaggtgtc

SEQ ID NO: 26 - protein, 423 aa, Sindbis virus
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA